SEQUENCE LISTING (19 sequences)

SEQ ID NO: 1 - protein, 29 aa, Homo sapiens
HSQGTFTSDYSKYLDSRRAQDFVQWLMNT

SEQ ID NO: 2 - protein, 37 aa, Homo sapiens
HSQGTFTSDYSKYLDSRRAQDFVQWLMNTKRNRNNIA

SEQ ID NO: 3 - protein, 42 aa, Homo sapiens
YAEGTFISDYSIAMDKIHQQDFVNWLLAQKGKKNDWKHNITQ

SEQ ID NO: 4 - protein, 30 aa, Artificial Sequence
  glucagon-like peptide 1, GLP-1 (7-36)
HAEGTFTSDVSSYLEGQAAKEFIAWLVKGR

SEQ ID NO: 5 - protein, 29 aa, Scyliorhinus canicula
HSEGTFTSDYSKYMDNRRAKDFVQWLMNT

SEQ ID NO: 6 - protein, 29 aa, Artificial Sequence
  Dogfish glucagon analogue Y1(D-A2) dogfish glucagon
YAEGTFTSDYSKYMDNRRAKDFVQWLMNT

SEQ ID NO: 7 - protein, 29 aa, Artificial Sequence
  Dogfish glucagon analogue (D-A2) dogfish glucagon
HAEGTFTSDYSKYMDNRRAKDFVQWLMNT

SEQ ID NO: 8 - protein, 29 aa, Artificial Sequence
  Dogfish glucagon analogue (Aib2) dogfish glucagon
HXEGTFTSDYSKYMDNRRAKDFVQWLMNT

SEQ ID NO: 9 - protein, 29 aa, Artificial Sequence
  Dogfish glucagon analogue (Abu2) dogfish glucagon
HXEGTFTSDYSKYMDNRRAKDFVQWLMNT

SEQ ID NO: 10 - protein, 38 aa, Artificial Sequence
  Dogfish glucagon analogue (D-A2) dogfish glucagon exendin end
HAEGTFTSDYSKYMDNRRAKDFVQWLMNTPSSGAPPPS

SEQ ID NO: 11 - protein, 29 aa, Artificial Sequence
  Dogfish glucagon analogue (D-A2I7) dogfish glucagon
HAEGTFISDYSKYMDNRRAKDFVQWLMNT

SEQ ID NO: 12 - protein, 29 aa, Artificial Sequence
  Dogfish glucagon analogue (D-A2I12) dogfish glucagon
HAEGTFTSDYSIYMDNRRAKDFVQWLMNT

SEQ ID NO: 13 - protein, 29 aa, Artificial Sequence
  Dogfish glucagon analogue (D-A2A13) dogfish glucagon
HAEGTFTSDYSKAMDNRRAKDFVQWLMNT

SEQ ID NO: 14 - protein, 29 aa, Artificial Sequence
  Dogfish glucagon analogue (D-A2D-Y13) dogfish glucagon
HAEGTFTSDYSKYMDNRRAKDFVQWLMNT

SEQ ID NO: 15 - protein, 29 aa, Artificial Sequence
  Dogfish glucagon analogue (D-A2D-D21) dogfish glucagon
HAEGTFTSDYSKYMDNRRAKDFVQWLMNT

SEQ ID NO: 16 - protein, 30 aa, Artificial Sequence
  Dogfish glucagon analogue (D-A2) dogfish glucagon Lys30-?-Glutamyl-PAL
HAEGTFTSDYSKYMDNRRAKDFVQWLMNTK

SEQ ID NO: 17 - protein, 29 aa, Artificial Sequence
  Dogfish glucagon analogue (D-A2) dogfish glucagon Lys20-?-Glutamyl-PAL
HAEGTFTSDYSKYMDNRRAKDFVQWLMNT

SEQ ID NO: 18 - protein, 29 aa, Artificial Sequence
  Dogfish glucagon analogue (D-A2) dogfish glucagon-Lys12-?-Glutamyl-PAL
HAEGTFTSDYSKYMDNRRAKDFVQWLMNT

SEQ ID NO: 19 - protein, 7 aa, Artificial Sequence
  Motif within dofish glucagon
MDNRRAK

(19) United States
(12) Patent Application Publication
(10) Pub. No.: US 2015/0197550 A1
(43) Pub. Date: Jul. 16, 2015

(54) Therapeutic Uses of Dogfish Glucagon and Analogues Thereof

(71) Applicant: UNIVERSITY OF ULSTER, Coleraine, C. Londonderry (GB)

(72) Inventors: Peter R. Flatt, Portrush (GB); Finbarr P.M. O'Harte, Portstewart (GB)

(73) Assignee: UNIVERSITY OF ULSTER, Coleraine, C. Londonderry (GB)

(21) Appl. No.: 14/421,405

(22) PCT Filed: Aug. 14, 2013
(86) PCT No.: PCT/EP2013/067043

§ 371 Date: Feb. 12, 2015

(30) Foreign Application Priority Data
Aug. 14, 2012 (GB) 1214493.7

(51) Int. Cl. C07K 14/605

(52) U.S. Cl. CPC C07K 14/605; A61K 38/00

(57) ABSTRACT

The present invention relates to peptides of 12 to 50 amino acids in length which incorporate the amino acid sequence MDNRRAK for use in the treatment of obesity, type-2 diabetes or metabolic syndrome.

The present invention relates to peptides for use in treating obesity or type-2 diabetes.

Type-2 diabetes is a chronic metabolic disorder resulting from a combination of impaired insulin secretion and impaired insulin action, the latter also known as insulin resistance. It can be recognized by hyperglycaemia and can lead to various microvascular complications such as retinopathy, neuropathy and nephropathy. Diabetes is the seventh leading cause of death in the USA with the most important causes of mortality in these patients being heart failure and stroke. Diabetogenic lifestyle factors such as a high fat diet and lack of exercise contribute to the rising prevalence of obesity, insulin resistance and diabetes; which in turn are associated with the increasing numbers of Type-2 diabetic patients. Obesity and in particular central obesity is a strong risk factor for developing Type-2 diabetes and the majority of such sufferers are overweight or obese, around 90% of cases, having a body mass index (BMI) of 25 kg/m² and above.

Type-2 diabetes is initially treated using oral anti-diabetic medication (otherwise known as “oral hypoglycaemic agents” or “oral antihyperglycaemic agents”. Such treatments include biguanides (such as metformin), sulphonylureas (such as tolbutamide, acetohexamide, tolazamide, chlorpropamide, glipizide, glibenclamide, glimepiride, gliclazide, glycopyramide and gliquidone), thiazolidinediones (such as rosiglitazone, pioglitazone and troglitazone), meglitinides (such as repaglinide and nateglinide) α-glucosidase inhibitors (such as miglitol, acarbose and voglibose) and dipeptyl peptidase-4 inhibitors (such as vildagliptan, sitagliptan, saxagliptan, linagliptin, allogliptin and septagliptin).

However, for many diabetic patients, the oral medication described above, both in isolation and in combination with one another, is unable to control the condition adequately. In advanced cases, the use of parenteral medication is required (in combination with the oral medication). Insulin is the main form of injectable diabetic therapy. Insulin is available in fast-acting formulations (used at meal times) and in sustained release formulations (used to provide base-line insulin). Injectable glucagon-like peptide-1 (GLP-1) analogues (such as exentatide, liraglutide, lixisenatide and taspoglutide) are another form of injectable antihyperglycemic agents.

With regard to obesity, there is only one medication approved by the MHRC used to treat the condition, orlistat. Orlistat inhibits gastric and pancreatic lipases, preventing ingested triglycerides from being hydrolysed into absorbable fatty acids so that they are excreted rather than absorbed. Even though orlistat has been shown to reduce weight significantly in patients, weight reduction is found to be minimal, and its unpleasant gastrointestinal side effects mean it is not a popular form of medication with all patients.

The above problems associated with obesity and type-2 diabetes mean there is a need for alternative treatments for these conditions. Clinically, treatments which reduced morbidity and mortality in these conditions would be desirable. Treatments which are safe and easy to administer would be particularly useful.

It is known that a number of peptide hormones play a role in metabolic regulation and potentially in the pathogenesis of diabetes.

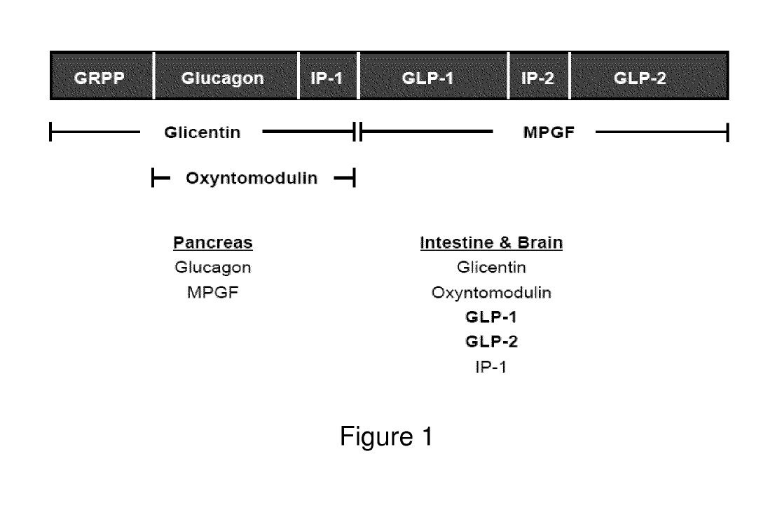

Proglucagon is an important gene involved in maintaining regulated processes in the body. A number of key peptides are processed from this gene such as glucagon in the pancreas, and glucagon-like peptide-1 (GLP-1) in the intestine and brain. FIG. 1 shows the structural organisation of mammalian proglucagon and all the proglucagon derived peptides. The proglucagon gene is expressed in the α-cells of the endocrine pancreas, the L-cells of the intestine, and the neurons located in the caudal brainstem and hypothalamus. This gene product is differentially processed in the pancreas and brain/gut with the main components produced differing in these tissues.

Glucagon, a 29 residue polypeptide hormone derived from the proglucagon gene, is important in the regulation of glucose homeostasis in the body. When it is secreted by the α-cells of the pancreas it binds to its specific receptors located in the liver plasma membrane. In type-2 diabetic patients, circulating glucagon levels are inappropriately high (hyperglucagonaemia) and insulin resistance intensifies this situation as insulin normally opposes glucagon action.

In addition to the classical effect of glucagon (raising blood glucose levels), acute glucagon administration reduces food intake in animals and in humans, however investigation of glucagon's metabolic effects are difficult since the endogenous hormone is rapidly degraded by dipeptidyl peptidase-4 (DPP-4).

Glucagon-like peptide-1 (GLP-1) is an incretin hormone. Stimulation of GLP-1 secretion from the intestinal endocrine L-cells, which are located primarily in the distal ileum and colon, is brought about by a number of nutrient, neural, and endocrine factors. The main physiologic stimulus of GLP-1 secretion from the L-cells is after meal ingestion, particularly one which is rich in fats and carbohydrates. The ubiquitous proteolytic enzyme DPP-4 rapidly inactivates GLP-1 in the circulation, giving GLP-1 a half-life of less than two minutes in man. The GLP-1 receptor (GLP-1R) has been identified and is expressed in a wide range of tissues including α-, β- and δ-cells of the pancreatic islets, lung, heart, kidney, stomach, intestine, pituitary, skin, nodose ganglion neurons of the vagus nerve, and several regions of the CNS including the hypothalamus and brainstem.

By improving both glucose-stimulated insulin synthesis and secretion at the pancreas, GLP-1 lowers glucose through multiple mechanisms. Adenylate cyclase activity and cAMP production are activated when GLP-1 binds to its specific receptor on pancreatic β-cells. Also, GLP-1 will work in unison with glucose to promote insulin gene transcription, mRNA stability, and biosynthesis. Therefore, this prevents an ability to replenish β-cell insulin stores and stop exhaustion of β-cell reserves. GLP-1 has also been shown to have central satiation-inducing properties, thus reducing food intake and a loss of body weight. Injectable GLP-1R agonists have been shown to decrease gastric motility and reduce post-meal glucose absorption.

Glucose-dependent insulinotropic polypeptide (GIP) is an incretin hormone first identified when it was isolated from crude extracts of porcine small intestine. It is a 42 amino acid peptide released from K-cells of the small intestine. GIP(1-42) is degraded into its inactive form GIP(3-42) by DPP-IV and has a half-life of less than 7 minutes in humans. Bioactive GIP is derived from its proGIP protein precursor. The primary physiologic role of GIP is an incretin hormone, it is secreted in response to nutrient ingestion, binds to its G-protein-coupled receptor on pancreatic β-cells, and enhances glucose-dependent insulin secretion.

The inventors have found that the glucagon derived from the common dogfish (SEQ ID NO:5) has surprising properties. The common dogfish (also known as the rough hound, bull huss, nurse hound, small-spotted catshark or lesser spotted dogfish) has the binominal name of Scyliorhinus canicula. Even though the sequence of the glucagon protein derived from this species is known, the effects of this protein in humans was not known. Surprisingly, the dogfish glucagon neither agonises nor antagonises the human GCGR, but instead agonises the human GIPR. As described above, such an agonist could have beneficial effects in treating type-2 diabetes or obesity.

The inventors also surprisingly found that modifications to the basic dogfish glucagon sequence can change the receptors that the peptide activates. For example, the substitution at position 2 from a serine residue to a D-isomer of alanine led to a peptide that neither agonises nor antagonises the human GIPR or the human GCGR, but instead agonises the human GLP-1R.

The inventors found the motif of Met-Asp-Asn-Arg-Arg-Ala-Lys or MDNRRAK (SEQ ID NO: 19), to be of particular importance in increasing insulin secretion in vitro and in vivo.

Thus the present invention provides a peptide of 12 to 50 amino acids in length which incorporates the amino acid sequence MDNRRAK for use in the treatment of obesity, type-2 diabetes or metabolic syndrome.

The inventors do not wish to speculate on a required mechanism of action. Nevertheless, in a preferred embodiment, the invention provides a peptide of 12 to 50 amino acids in length which incorporates the amino acid sequence MDNRRAK and can agonise either or both of the human GIP and GLP-1 receptors, for use in the treatment of obesity, type-2 diabetes or metabolic syndrome.

In certain preferred embodiments the peptide also interacts with the GCGR, preferably it is a GCGR agonist. In certain particularly preferred embodiments, the peptide is an agonist of both GIPR and GLP-1R.

Alternatively viewed, the invention provides a method of treating obesity, type-2 diabetes or metabolic syndrome, which method comprises administration to a subject in need thereof of a therapeutically effective amount of a peptide of 12 to 50 amino acids in length which incorporates the amino acid sequence MDNRRAK. A therapeutically effective amount will be determined based on the clinical assessment and can be readily monitored, in vivo, in relation to the hormones involved in regulation of glucose levels or glucose itself e.g. insulin levels.

Alternatively viewed, the invention provides use of a peptide of 12 to 50 amino acids in length which incorporates the amino acid sequence MDNRRAK in the manufacture of a medicament for the treatment of obesity, type-2 diabetes or metabolic syndrome.

In preferred embodiments of the invention, the peptides of the invention are of 20 to 45 amino acids in length, preferably 25 to 40, e.g. 27 to 32, in particular 29 amino acids in length.

The peptides of use in the invention are preferably dogfish glucagon or analogues thereof. Thus the peptides preferably comprise or consist of a region of sequence homology with dogfish glucagon (1-29) (SEQ ID No. 5). Preferably the primary amino acid sequence of this homologous sequence has no more than 8, or 6 more preferably no more than 4, e.g. 1 to 3 amino acids which are added, deleted or substituted as compared to the native dogfish glucagon sequence. Replacement of an L form of an amino acid with a D form does not constitute a “substitution” as defined herein. However, if an amino acid side chain is modified this is a substitution.

The peptides of the invention comprise the heptapeptide defined above and typically have flanking N and C terminal regions, for example, each flanking region may consist of 5 to 20 amino acids, preferably the N terminal flanking region consists of 5 to 15 amino acids, more preferably 10 to 15 amino acids and the C terminal flanking region consists of 4 to 10 amino acids, more preferably 6 to 9 amino acids. These flanking regions will typically have the sequence of part of native dogfish glucagon, optionally with a limited number of additions, deletions or substitutions, as defined above.

Preferred modifications as compared to the native dogfish glucagon sequence are modifications to increase in vivo stability, e.g. resistance to degradation by enzymes. Such modifications are described in more detail below. Alternative modifications include substitutions designed to target either the GIP or GLP-1 receptor by introducing equivalent residues to the native target sequence for those receptors; for example substitution at positions 7, 12, or 13 of the native sequence with isoleucine (7, 12) or alanine (13), these residues make the peptide more ‘GIP-like’. Position 2 is a particularly preferred location for modification, e.g. by introduction of Ala, D-Ala or a non-genetically coded amino acid such as Aib or Abu. Position 1 is a further preferred position for modification, e.g. for replacement with Tyr.

In accordance with the present invention, “treatment” includes improving one or more of the symptoms of the condition and reducing or preventing the long term complications associated with the conditions. The condition “type-2 diabetes” is also known as “non-insulin dependent diabetes mellitus”, “NIDDM” and “adult-onset diabetes”. This condition is often caused by an inability of cells to adequately respond to normal levels of insulin. However, the peptides of the invention may also be of benefit in type-1 (insulin dependent) diabetes mellitus or gestational diabetes mellitus. “The treatment of type-2 diabetes” includes reducing, or reducing the risk of, the secondary complications associated with type-2 diabetes (such as cardiovascular disease, diabetic retinopathy, chronic nephropathy, peripheral neuropathy), and thus improving the symptoms and signs associated with type-2 diabetes.

The therapies proposed will preferably result in improvement in one or more of the following: insulin resistance, abdominal obesity and hyperglycemia, in particular in patients diagnosed with type-2 diabetes and/or classified as obese. Obesity includes any condition whereby excessive levels of body fat have adverse effects on health. Obesity is often diagnosed as having a body mass index (BMI) of 30 kg/m² or greater and morbid obesity as BMI>40 kg/m2 (see Kee et al. 2012 Obes Rev. 2012 May 8. doi: 10.1111/j.1467-789X.2012.01000.x). The peptides of the invention may be of benefit to patients who are considered as “overweight” (a BMI of 25 to 30 kg/m²) as well as obese and morbidly obese patients.

The term “obesity” also includes the condition “central obesity” (otherwise known as “belly fat” or “abdominal obesity”), where there is a specific increase in abdominal fat compared to fat in other areas.

“The treatment of obesity” means not only the reduction in body fat (leading to a reduction in BMI) but also reduction in or prevention of the secondary complications associated with obesity, such as cardiovascular disease, osteoarthritis, obstructive sleep apnoea, cancer, non-alcoholic fatty liver disease, as well as type-2 diabetes.

“Metabolic syndrome” is a combination of medical disorders that, when occurring together, increase the risk of developing cardiovascular disease and diabetes. Some studies have shown that the prevalence in the USA to be approximately 25% of the population, and the prevalence increases with age. Metabolic syndrome is also known as metabolic syndrome X, cardiometabolic syndrome, syndrome X, insulin resistance syndrome, Reaven's syndrome, and CHAOS.

The “treatment of metabolic syndrome” means not only a reduction in the signs used to diagnose the condition (such as raised BMI, impaired glucose tolerance, increased insulin resistance, increased blood pressure, dyslipidaemia and microalbuminuria) but also a reduction in the secondary complications associated with metabolic syndrome, such as cardiovascular disease and diabetes, as well as a reduction in the risk of developing these complications.

The subjects to be treated are preferably human but the treatments may also be applied to other mammals, in particular companion animals or livestock, e.g. dogs, cats, horses, cows. Likewise, unless otherwise clear from the context, reference to the glucagon (GCG), GIP or GLP-1 receptor is to the human receptor.

The peptides of the invention may be modified in order to increase the stability of the peptide in vivo. Such modifications include, but are not limited to, the substitution of an L form amino acid with a D-isomer equivalent. N and particularly C terminal modifications, e.g. amidation of the C terminus, may provide an alternative route to improving in vivo stability.

Modifications may also involve the inclusion of non-genetically coded amino acids, such as 2-methyl alanine (otherwise known as 2-aminoisobutyric acid, α-aminoisobutyric acid, α-methyl alanine or Aib) and 2-aminobutyric acid (Abu).

Another suitable modification is the attachment of a lipophilic moiety to one or more lysine residues. The peptide may have a lipophilic moiety attached to more than one of the lysine residues contained therein but preferably only one lysine residue will be so modified. The lysine residue (K) in the MDNRRAK motif may be modified in this way. Preferably, the lysine residue at position 30, 20 or 12 of the native dogfish glucagon sequence, preferably the lysine at position 30, is modified in this way.

The lysine residue(s) is preferably substituted with a lipophilic substituent at the ε-amino group. A carboxyl group of the lipophilic substituent may form an amide bond with the ε-amino group. The lipophilic substituent preferably comprises 8-24 carbon atoms. Preferably the lipophilic substituent is derived from a fatty acid which may be saturated or unsaturated, preferably saturated. Suitably fatty acids include: lauric, myristic, palmitic, stearic, behenic, palmitoleic, oleic, linoleic, α-linoleic, γ-linoleic and arachidonic acid.

In a preferred embodiment the lipophilic substituent may be attached to the lysine residue by a spacer, for example in such a way that a carboxyl group of the spacer forms an amide bond with the ε-amino group of lysine. In a preferred embodiment, the spacer is an α,ω-amino acid. Examples of suitable spacers are succinic acid, Lys, Glu or Asp, or a dipeptide such as Gly-Lys. When the spacer is succinic acid, one carboxyl group thereof may form an amide bond with the amino group of lysine, and the other carboxyl group thereof may form an amide bond with an amino group of the lipophilic substituent. When the spacer is Lys, Glu or Asp, the carboxyl group thereof may form an amide bond with the amino group of lysine, and the amino group thereof may form an amide bond with a carboxyl group of the lipophilic substituent. In another preferred embodiment such a further spacer is Glu or Asp which forms an amide bond with the ε-amino group of Lys and another amide bond with a carboxyl group present in the lipophilic substituent, that is, the lipophilic substituent is a N^{ε}-acylated lysine residue. Other preferred spacers are N^{ε}-(γ-L-glutamyl), N^{ε}-(β-L-asparagyl), N^{ε}-glycyl, and N^{ε}-(α-(γ-aminobutanoyl). N^{ε}-(γ-L-glutamyl), (gamma glutamyl) is particularly preferred as a spacer.

In another preferred embodiment of the present invention, the lipophilic substituent has a group which can be negatively charged. One preferred such group is a carboxylic acid group. In a further preferred embodiment, the lipophilic substituent is attached to the parent peptide by means of a spacer which is an unbranched alkane, e.g. α,Ω-dicarboxylic acid group having from 1 to 7 methylene groups, preferably two methylene groups which spacer forms a bridge between the E-amino group of lysine and an amino group of the lipophilic substituent. Further suitable spacers are described in U.S. Pat. No. 6,268,343 the teaching of which is incorporated herein by reference.

Thus the lipophilic substituent may be an acyl group of formula CH₃(CH₂)ₙCO— wherein n is an integer from 6 to 38, preferably an integer from 6 to 22, e.g. CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— or CH₃(CH₂)₂₂CO—.

In a further preferred embodiment, the lipophilic substituent is an acyl group of a straight-chain or branched alkane, α,ω-dicarboxylic acid.

In a further preferred embodiment, the lipophilic substituent is an acyl group of the formula HOOC(CH₂)ₘCO—, wherein m is an integer from 6 to 38, preferably an integer from 6 to 24, more preferably HOOC(CH₂)₁₄CO—, HOOC(CH₂)₁₆CO—, HOOC(CH₂)₁₈CO—, HOOC(CH₂)₂₀CO— or HOOC(CH₂)₂₂CO—. Preferably, the lipophilic substituent is a γ-glutamyl-palmitate group (γ-glutamyl-PAL).

Without wishing to be bound by theory, the lipophilic substituents may self-aggregate and/or non-covalently bind to plasma albumin fatty acid binding sites. This results in slower absorption and prolongs the half-life of the peptides.

Preferred peptides of the invention are set out in Table 2 herein and from include: dogfish glucagon (SEQ ID NO:5), Y¹ (D-A²) dogfish glucagon (SEQ ID NO:6), (D-A²) dogfish glucagon (SEQ ID NO:7), (Aib²) dogfish glucagon (SEQ ID NO:8), (Abu-A²) dogfish glucagon (SEQ ID NO:9), (D-A²) dogfish glucagon exendin end (SEQ ID NO:10), (D-A²I⁷) dogfish glucagon (SEQ ID NO:11), (D-A²I¹²) dogfish glucagon (SEQ ID NO:12), (D-A²A¹³) dogfish glucagon (SEQ ID NO:13), (D-A²D-Y¹³) dogfish glucagon (SEQ ID NO:14), (D-A²D-D²¹) dogfish glucagon (SEQ ID NO:15), (D-A²) dogfish glucagon Lys³⁰γ-Glutamyl-PAL (SEQ ID NO:16), (D-A²) dogfish glucagon Lys²⁰γ-Glutamyl-PAL (SEQ ID NO:17) and (D-A²) dogfish glucagon Lys¹²γ-Glutamyl-PAL (SEQ ID NO:18).

Particularly preferred are dogfish glucagon (SEQ ID NO:5) or (D-A²) dogfish glucagon (SEQ ID NO:7) and Y¹ (D-A²) dogfish glucagon (SEQ ID NO:6).

Also particularly preferred are dogfish glucagon (SEQ ID NO:5) and (D-A²) dogfish glucagon (SEQ ID NO:7) and Y¹ (D-A²) dogfish glucagon (SEQ ID NO:6) and (D-A²) dogfish glucagon Lys³⁰γ-Glutamyl-PAL (SEQ ID NO:16).

Particularly preferred peptides are (D-A²) dogfish glucagon (SEQ ID NO:7) and (D-A²) dogfish glucagon Lys³⁰γ-Glutamyl-PAL (SEQ ID NO:16), most preferably (D-A²) dogfish glucagon (SEQ ID NO:7).

In a further aspect, the present invention provides a peptide of 12 to 50 amino acids in length which incorporates the amino acid sequence MDNRRAK which is capable of stimulating insulin secretion, in particular which is capable of acting as an agonist to the human GIP or GLP-1 receptor, excluding the peptide of SEQ ID NO. 5. Preferred amongst these peptides are those discussed herein as preferred in the context of the treatment of type-2 diabetes and obesity.

Methods for assessing the ability of a molecule to stimulate insulin secretion are known in the art and the effect may be in vivo and/or in vitro. The Examples provide a convenient method to assess insulin secretion utilising BRIN-BD11 cells. The analogues preferably exhibit at least 30%, more preferably at least 50% or at least 70%, most preferably at least 90% or at least 100%, of the ability of native dogfish glucagon to stimulate insulin secretion.

The peptides of the invention may either be modified using the isolated dogfish glucagon peptide as a starting point, or may be synthesised de novo in any convenient way. Generally the reactive groups present (for example amino, thiol and/or carboxyl) will be protected during modification or synthesis. The final step in the synthesis will thus be the deprotection of a protected derivative of the invention.

With regard to synthesising the peptide, one can in principle start either at the C-terminal or the N-terminal although the C-terminal starting procedure is preferred as this is the normal procedure applied in fMoc solid phase peptide synthesis.

Methods of peptide synthesis are well known in the art and include recombinant DNA technology but for the present invention it may be particularly convenient to carry out the synthesis on a solid phase support, such supports being well known in the art.

A wide choice of protecting groups for amino acids are known and suitable amine protecting groups may include carbobenzyloxy (Z) t-butoxycarbonyl (Boc), 4-methoxy-2,3,6-trimethylbenzene sulphonyl (Mtr) and 9-fluorenylmethoxy-carbonyl (Fmoc). It will be appreciated that when the peptide is built up from the C-terminal end, an amine-protecting group will be present on the α-amino group of each new residue added and will need to be removed selectively prior to the next coupling step.

Carboxyl protecting groups which may, for example be employed include readily cleaved ester groups such as benzyl (Bzl), p-nitrobenzyl (ONb), or t-butyl (OtBu) groups as well as the coupling groups on solid supports, for example the Rink amide linked to polystyrene.

Thiol protecting groups include p-methoxybenzyl (Mob), trityl (Trt) and acetamidomethyl (Acm).

Preferred peptides of the invention may conveniently be prepared using the t-butyloxycarbonyl (Boc) protecting group for the amine side chains of Lys, Orn, Dab and Dap as well as for protection of the indole nitrogen of the tryptophan residues. Fmoc can be used for protection of the alpha-amino groups. For peptides containing Arg, 2,2,4,6,7-pentamethyldihydrobenzofurane-5-sulfonyl can be used for protection of the guanidine side chain.

A wide range of procedures exists for removing amine- and carboxyl-protecting groups. These must, however, be consistent with the synthetic strategy employed. The side chain protecting groups must be stable to the conditions used to remove the temporary α-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

The peptides of the invention may be in the form of esters, amides or salts. In a further aspect are provided peptidomimetic equivalents of the peptides defined herein. A peptidomimetic is typically characterised by retaining the polarity, three-dimensional size and functionality of its peptide equivalent but wherein the peptide bonds have been replaced, often by more stable linkages. By ‘stable’ is typically meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. Suitable amide bond surrogates include the following groups: N-alkylation (Schmidt, R. et al., Int. J. Peptide Protein Res., 1995, 46,47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

Suitable peptidomimetics include reduced peptides where the amide bond has been reduced to a methylene amine by treatment with a reducing agent e.g. borane or a hydride reagent such as lithium aluminium hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA 1994, 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes.

While it is possible for the compounds of the present invention to be administered as pure compounds, it is preferable to present them as pharmaceutical compositions. Thus, pharmaceutical compositions according to the present invention preferably comprise at least one peptide or peptidomimetic as defined above, together with at least one other therapeutic ingredient. Thus, the present invention extends to the use of a pharmaceutical composition incorporating the compounds of the present invention and at least one other therapeutic ingredient.

Pharmaceutical compositions comprising the peptides described herein, with the exclusion of native dogfish glucagon, constitute a further aspect of the present invention. As does a method for the preparation of such peptides.

Methods of treating type-2 diabetes or obesity which comprise administration to a human or animal patient one or more of the peptides as defined herein constitute a further aspect of the present invention. These treatments may involve co-administration with another antidiabetic agent, e.g. insulin or an incretin or mimic or analogue thereof or antiobesity agent.

The compositions according to the invention may be presented, for example, in a form suitable for oral, topical, nasal, parenteral, intravenous, intratumoral, rectal or regional (e.g. isolated limb perfusion) administration. Administration is typically by a parenteral route, preferably by injection into the peritoneum, or subcutaneously, intramuscularly, intracapsularly, intraspinaly, intratumouraly or intravenously.

The active compounds defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Organ specific carrier systems may also be used.

Injection solutions may, for example, be produced in the conventional manner, such as by the addition of preservation agents, such as p-hydroxybenzoates, or stabilizers, such as EDTA. The solutions are then filled into injection vials or ampoules.

The peptide/peptides of the invention should be able to improve the severity of the condition as determined using diagnostic measures of either type-2 diabetes or obesity. Type-2 diabetes is diagnosed by recurrent or persistent hyperglycaemia. In humans, this is considered to be either (i) a blood glucose level of greater than 7.8 mmol/l (or greater than 140 mg/dl) 2 hours after glucose ingestion, (ii) a fasting blood glucose level of greater than 6.1 mmol/l (or greater than 110 mg/dl) or (iii) a glycated haemoglobin level of greater that 6%. The amount of the peptide/peptides of the invention administered should therefore be effective in reducing the levels of parameters (i-iii) if the peptide/peptides are being using to treat type-2 diabetes.

As described above, obesity is often measured by making a comparison of the patient's weight against the patient's height (body mass index BMI weight in Kg divided by height in meters squared). Obesity can also be measured through determining the patient's waist circumference (a waist circumference of greater than 102 cm in men or 88 cm in women is considered obese). The amount of the peptide/peptides of the invention administered should therefore be effective in reducing the weight (and therefore reduce the BMI) and/or waist circumference of the patient if the peptide/peptides are being using to treat obesity.

Dosage units containing the active molecules preferably contain 1 to 250 nmol/kg, for example 5 to 150 nmol/kg of the peptide/peptides of the invention. The pharmaceutical compositions may additionally comprise further active ingredients, including other antihyperglycaemic or anti-obesity agents.

Compounds of the invention and compounds suitable for the methods and uses of the invention include salt forms and appropriate pharmaceutically acceptable salts for peptides and similar molecules are well known to those skilled in the art.

The invention is further described in the following Examples, which includes some reference molecules outside the scope of the present invention, and with reference to the figures and tables in which:

FIG. 1 shows the structural organisation of mammalian proglucagon and all the proglucagon derived peptides. The proglucagon gene is expressed in the α-cells of the endocrine pancreas, the L-cells of the intestine, and neurons located in the caudal brainstem and hypothalamus. This gene product is differentially processed in the pancreas and brain/gut with the main components produced differing in these tissues. GRPP: glicentin-related polypeptide; IP-1 and IP-2: intervening peptide-1 and -2; MPGF: major proglucagon fragment; GLP-1 and GLP-2: glucagon-like peptide-1 and -2.

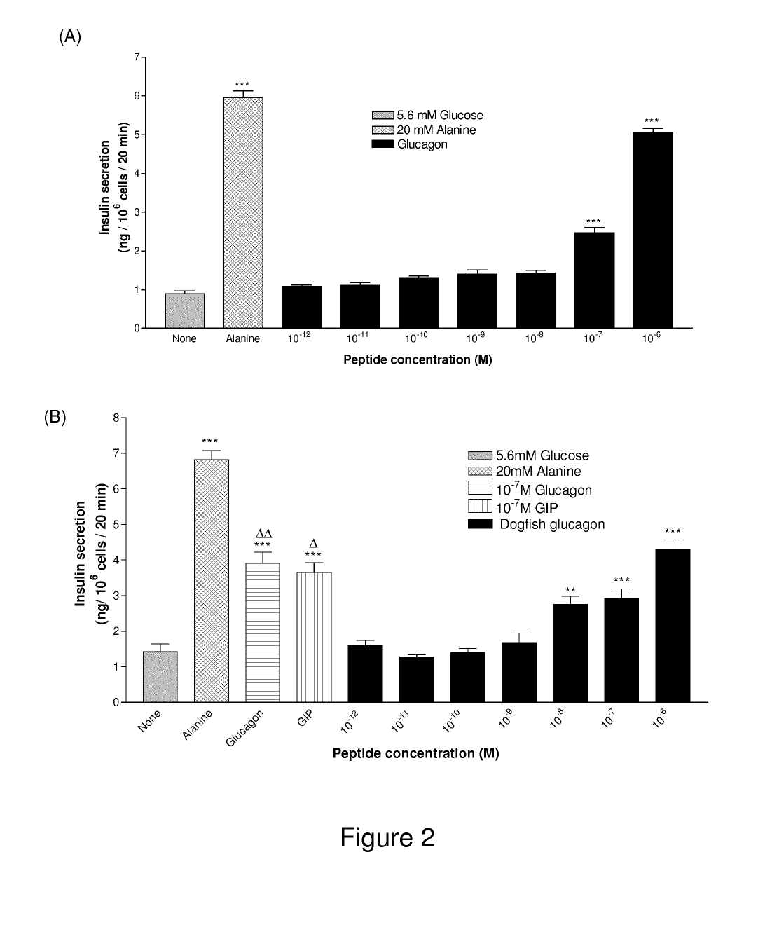

FIG. 2 shows the acute effects of increasing concentrations of human glucagon (SEQ ID NO:1) (A) and dogfish glucagon (SEQ ID NO:5) (B) on insulin secretion from BRIN-BD11 clonal β cells in the presence of 5.6 mM glucose for 20 min and insulin release measured using radioimmunoassay. Values represent means±SEM (n=8) where, **p<0.01 and ***p<0.001 compared with 5.6 mM glucose alone, ^{Δ}p<0.05 and ^{ΔΔ}p<0.01 compared to 10⁻⁷M dogfish glucagon (B).

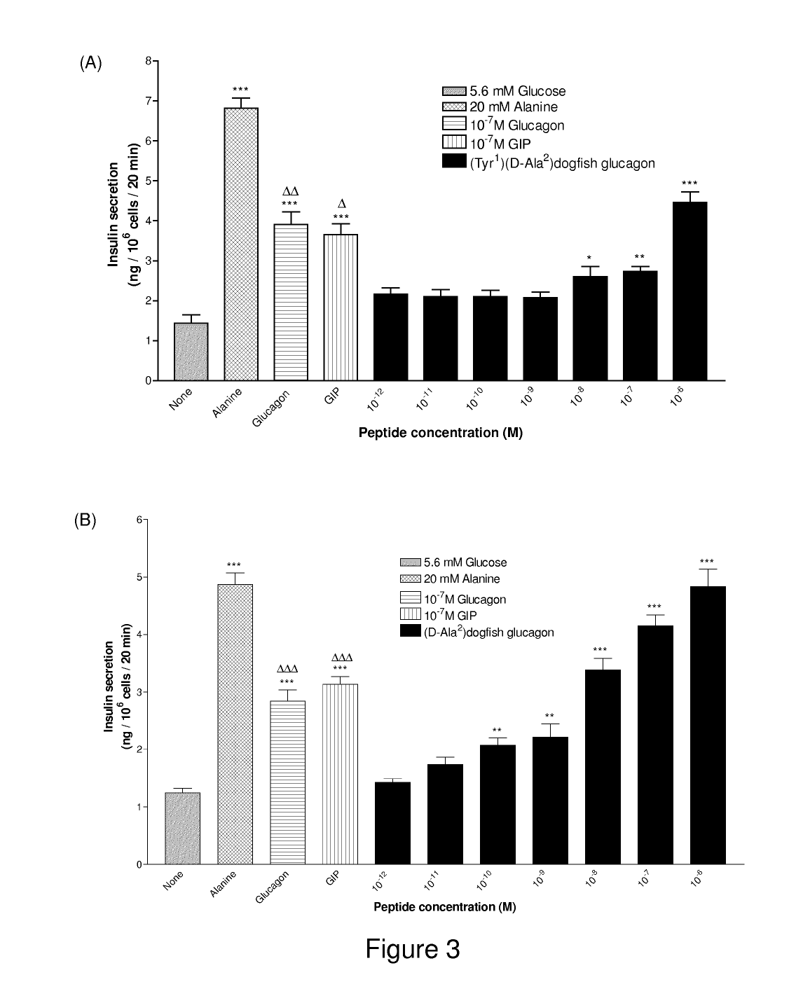

FIG. 3 shows the acute effects of increasing concentrations of (Tyr¹)(D-Ala²) dogfish glucagon (SEQ ID NO:6) (A) and (D-Ala²) dogfish glucagon (SEQ ID NO:7) on insulin secretion from BRIN-BD11 clonal β cells in the presence of 5.6 mM glucose for 20 min and insulin release measured using radioimmunoassay. Values represent means±SEM (n=8) where *p<0.05, **p<0.01 and ***p<0.001 compared with 5.6 mM glucose alone, ^{Δ}p<0.05, ^{ΔΔ}p<0.01 ^{ΔΔΔ}p<0.001 compared to 10⁻⁷M (Tyr¹)(D-Ala²) dogfish glucagon (A) or 10⁻⁷M (D-Ala²) dogfish glucagon (B).

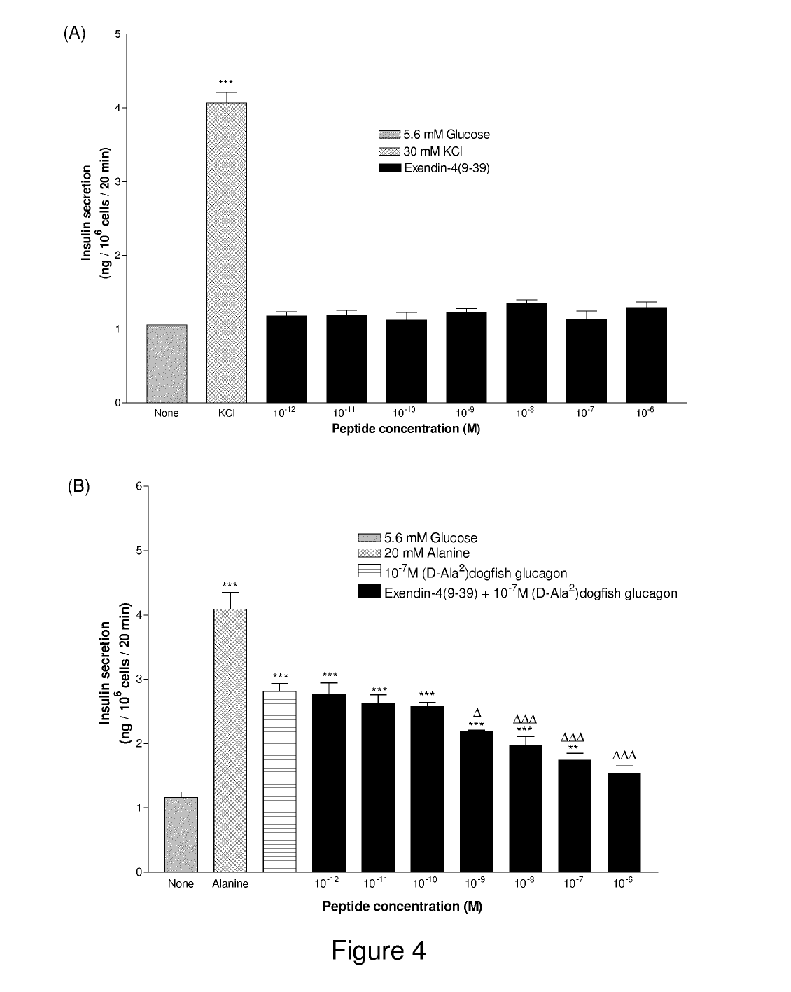

FIG. 4 shows the acute effects of increasing concentrations of Exendin-4(9-39) (a GLP-1R antagonist) alone (A) and increasing concentrations of Exendin-4(9-39) in the presence of 10⁻⁷M (D-Ala²) dogfish glucagon (B) (SEQ ID NO:7) on insulin secretion from BRIN-BD11 clonal β cells in the presence of 5.6 mM glucose for 20 min and insulin release measured using radioimmunoassay. Values represent means±SEM (n=8) where **p<0.01 and ***p<0.001 compared with 5.6 mM glucose alone, ^{Δ}p<0.05 and ^{ΔΔΔ}p<0.001 compared to 10⁻⁷M (D-Ala²) dogfish glucagon (B).

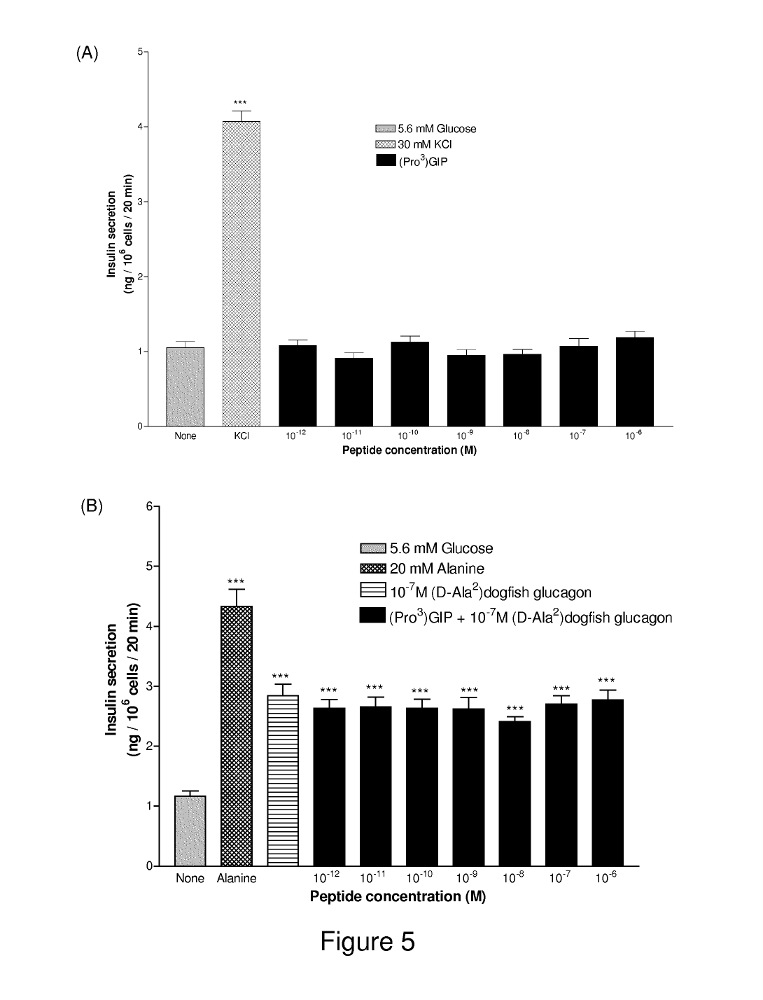

FIG. 5 shows the acute effects of increasing concentrations of (Pro³) GIP (a GIP antagonist) alone (A) and increasing concentrations of (Pro³) GIP in the presence of 10⁻⁷M (D-Ala²) dogfish glucagon (B) (SEQ ID NO:7) on insulin secretion from BRIN-BD11 clonal β cells in the presence of 5.6 mM glucose for 20 min and insulin release measured using radioimmunoassay. Values represent means±SEM (n=8) where ***p<0.001 compared with 5.6 mM glucose alone.

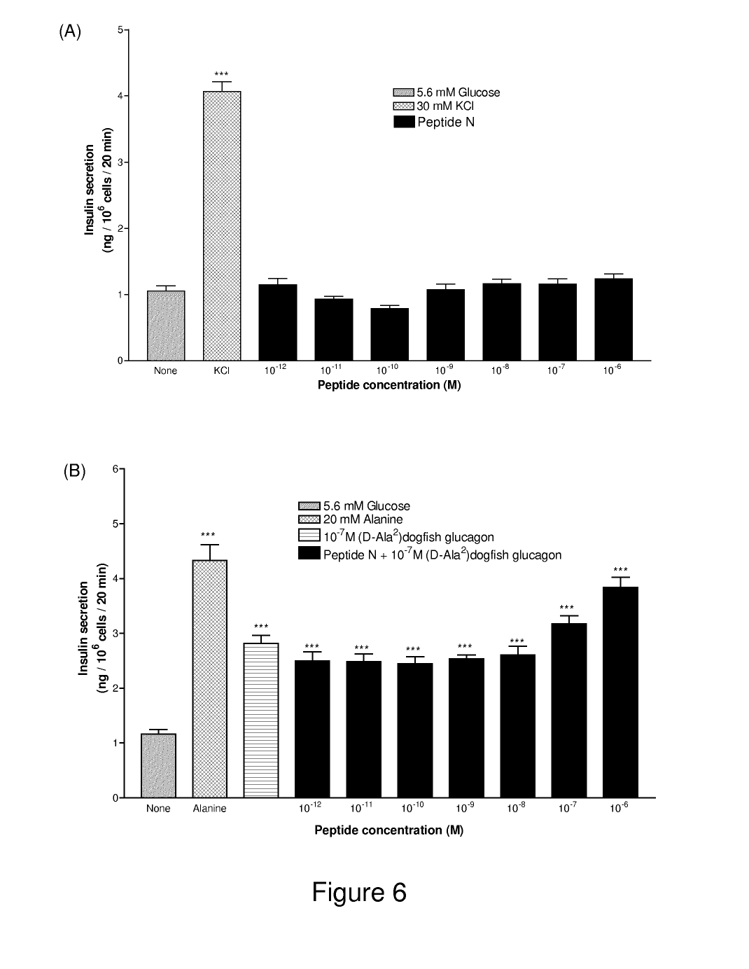

FIG. 6 shows the acute effects of increasing concentrations of Peptide N (also known as (desHis¹) (Pro⁴) glucagon, a GCGR antagonist) alone (A) and increasing concentrations of (desHis¹)(Pro⁴) glucagon in the presence of 10⁻⁷M (D-Ala²) dogfish glucagon (B) (SEQ ID NO:7) on insulin secretion from BRIN-BD11 clonal β cells in the presence of 5.6 mM glucose for 20 min and insulin release measured using radioimmunoassay. Values represent means±SEM (n=8) where ***p<0.001 compared with 5.6 mM glucose alone.

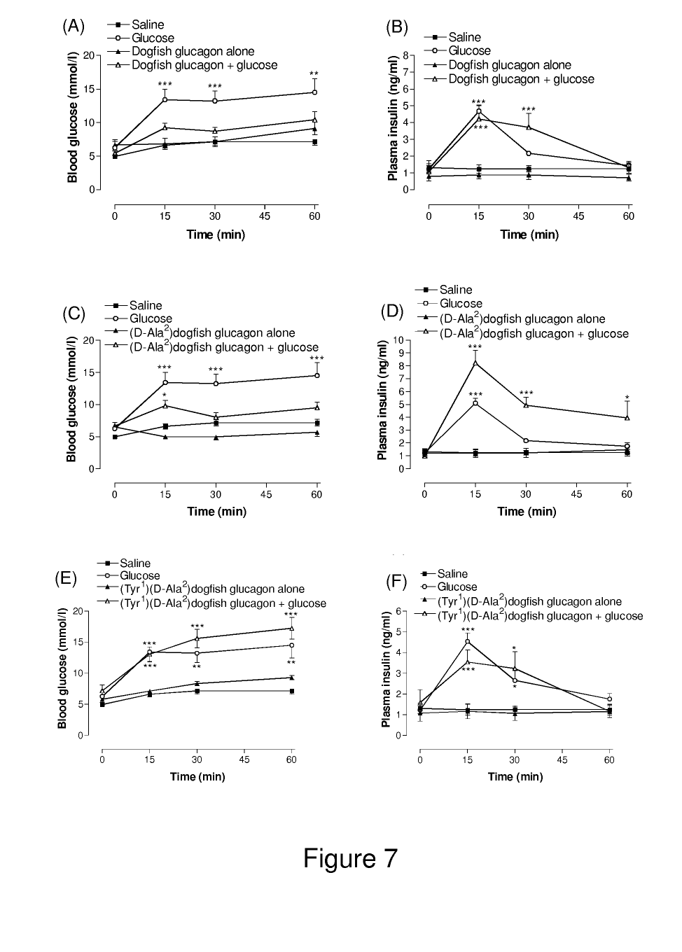

FIG. 7 shows the acute effects of dogfish glucagon (SEQ ID NO:5) (A&B), (D-Ala²) dogfish glucagon (SEQ ID NO:7) (C&D) and (Tyr¹)(D-Ala²) dogfish glucagon (SEQ ID NO:6) (E&F) in Swiss NIH mice. Blood glucose concentrations (A,C&E) and insulin concentrations (B,D&F) were measured prior to and after intraperitoneal administration of either (i) saline, (ii) one of dogfish glucagon, (D-Ala²) dogfish glucagon or (Tyr¹)(D-Ala²) dogfish glucagon alone (25 nmol/kg body weight), (iii) glucose alone (18 mmol/kg body weight) or (iv) glucose in combination with one of dogfish glucagon, (D-Ala²) dogfish glucagon or (Tyr¹)(D-Ala²) dogfish glucagon (25 nmol/kg body weight). Data are expressed as means±SEM for 8 mice, *p<0.05, **p<0.01 and ***p<0.001 compared to saline alone.

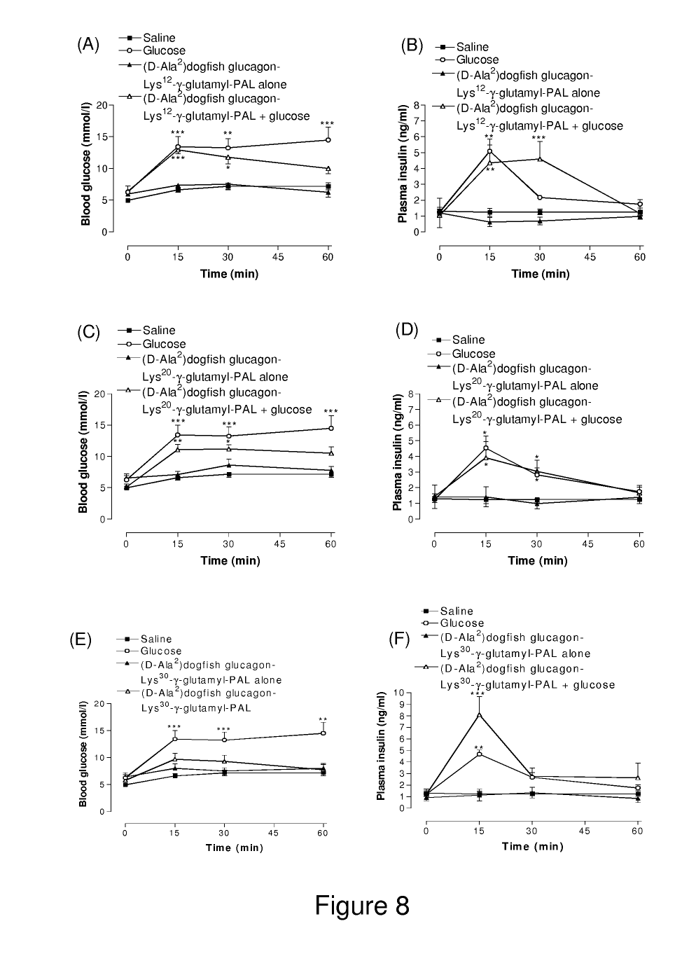

FIG. 8 shows the acute effects of (D-Ala²) dogfish glucagon-Lys¹²-γ-glutamyl-PAL (SEQ ID NO:18) (A&B), (D-Ala²) dogfish glucagon-Lys²⁰-γ-glutamyl-PAL (SEQ ID NO:17) (C&D) and (D-Ala²) dogfish glucagon-Lys³⁰-γ-glutamyl-PAL (SEQ ID NO:16) (E&F) in Swiss N/H mice. Blood glucose concentrations (A,C&E) and insulin concentrations (B,D&F) were measured prior to and after intraperitoneal administration of either (i) saline, (ii) one of (D-Ala²) dogfish glucagon-Lys¹²-γ-glutamyl-PAL, (D-Ala²) dogfish glucagon-Lys²⁰-γ-glutamyl-PAL or (D-Ala²) dogfish glucagon-Lys³⁰-γ-glutamyl-PAL alone (25 nmol/kg body weight), (iii) glucose alone (18 mmol/kg body weight) or (iv) glucose in combination with one of (D-Ala²) dogfish glucagon-Lys¹²-γ-glutamyl-PAL, (D-Ala²) dogfish glucagon-Lys²⁰-γ-glutamyl-PAL or (D-Ala²) dogfish glucagon-Lys³⁰-γ-glutamyl-PAL (25 nmol/kg body weight). Data are expressed as means±SEM for 8 mice, *p<0.05, **p<0.01 and ***p<0.001 compared to saline alone.

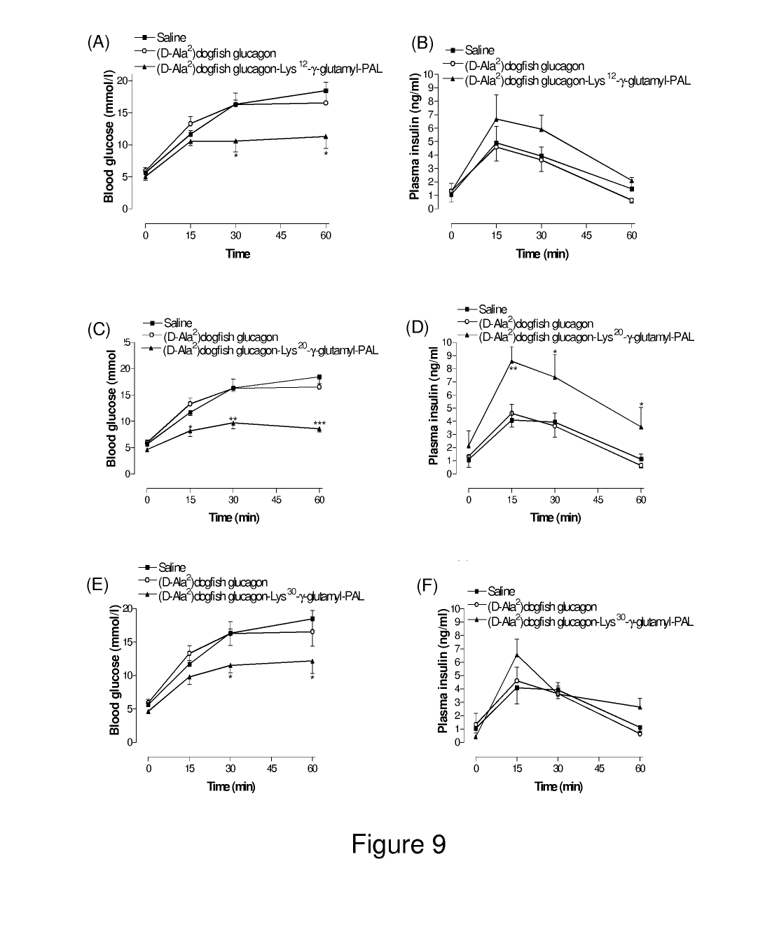

FIG. 9 shows the longer-term effects of (D-Ala²) dogfish glucagon-Lys¹²-γ-glutamyl-PAL (SEQ ID NO:18) (A&B), (D-Ala²) dogfish glucagon-Lys²⁰-γ-glutamyl-PAL (SEQ ID NO:17) (C&D) and (D-Ala²) dogfish glucagon-Lys³⁰-γ-glutamyl-PAL (SEQ ID NO:16) (E&F) in Swiss N/H mice. Blood glucose concentrations (A,C&E) and insulin concentrations (B,D&F) were measured after a glucose load (18 mmol/kg body weight) 4 hours after intraperitoneal administration of either (i) saline, (ii) (D-Ala²) dogfish glucagon (SEQ ID NO:7) (iii) one of (D-Ala²) dogfish glucagon-Lys¹²-γ-glutamyl-PAL, (D-Ala²) dogfish glucagon-Lys²⁰-γ-glutamyl-PAL or (D-Ala²) dogfish glucagon-Lys³⁰-γ-glutamyl-PAL alone (25 nmol/kg body weight). Food was removed at t=0. Data are expressed as means±SEM for 8 mice, *p<0.05, **p<0.01 and ***p<0.001 compared to saline alone.

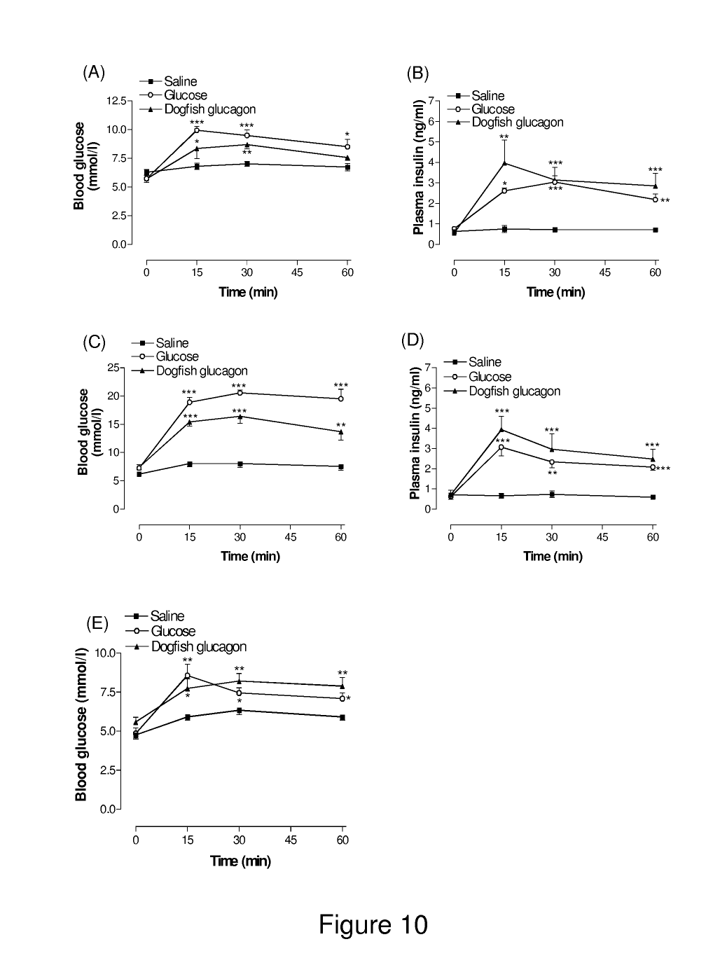

FIG. 10 shows the acute effects of dogfish glucagon (SEQ ID NO:5) on blood in C57 control mice (A&B), GLP-1R-KO mice (C&D) and GIPR-KO mice (E). Blood glucose concentrations (A,C&E) and insulin concentrations (B&D) were measured prior to and after intraperitoneal administration of either (i) saline, (ii) glucose alone (18 mmol/kg bw) or (iii) glucose in combination with dogfish glucagon (25 nmol/kg body weight). Data are expressed as means±SEM for 8 mice. *p<0.05, **p<0.01 and ***p<0.001 compared to saline alone.

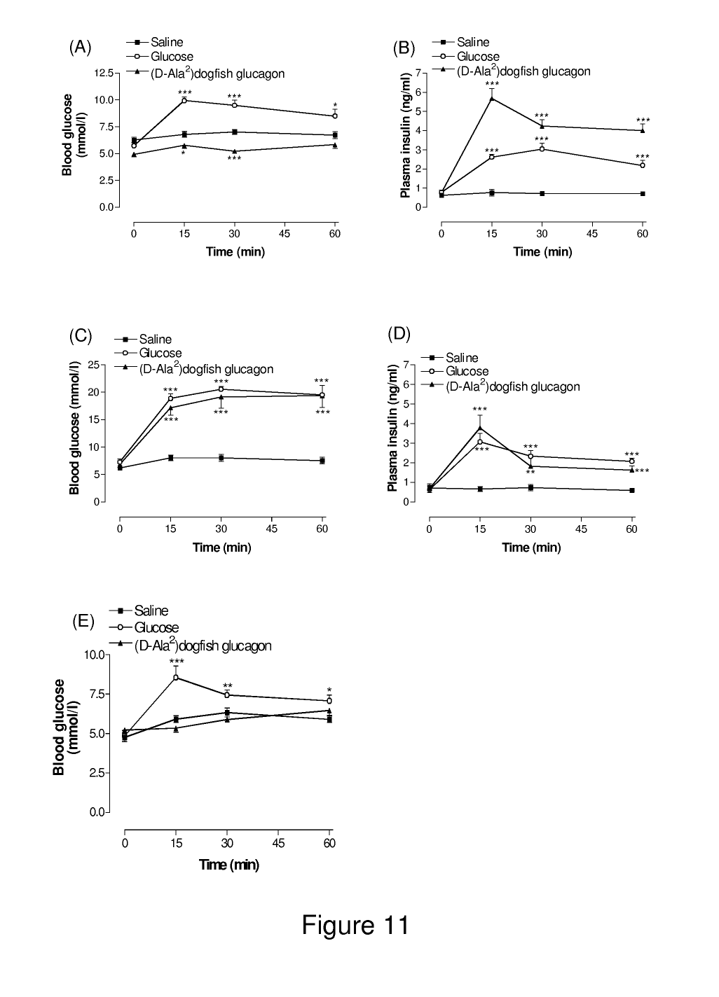

FIG. 11 shows the acute effects of (D-Ala²) dogfish glucagon (SEQ ID NO:7) on blood in C57 control mice (A&B), GLP-1R-KO mice (C&D) and GIPR-KO mice (E). Blood glucose concentrations (A,C&E) and insulin concentrations (B&D) were measured prior to and after intraperitoneal administration of either (i) saline, (ii) glucose alone (18 mmol/kg bw) or (iii) glucose in combination with dogfish glucagon (25 nmol/kg body weight). Data are expressed as means±SEM for 8 mice. *p<0.05, **p<0.01 and ***p<0.001 compared to saline alone.

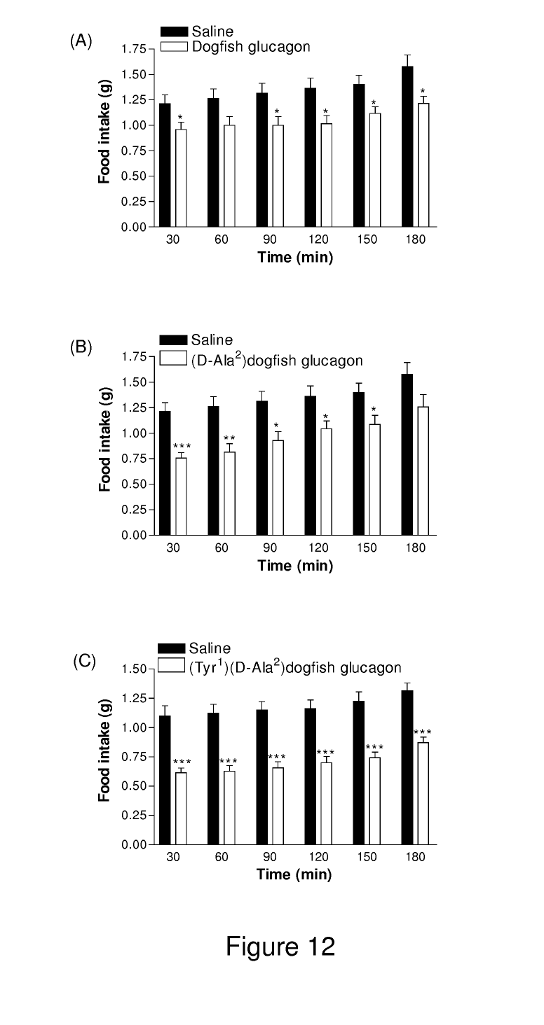

FIG. 12 shows the acute effects of dogfish glucagon (SEQ ID NO:5) (A), (D-Ala²) dogfish glucagon (SEQ ID NO:7) (B) and (Tyr¹)(D-Ala²) dogfish glucagon (SEQ ID NO:6) (C) on cumulative food intake in Swiss NIH mice. Food intake was measured in animals immediately following intraperitoneal injection with saline (0.9% (w/v), dogfish glucagon, (D-Ala²) dogfish glucagon or (Tyr¹)(D-Ala²) dogfish glucagon (each at 100 nmol/kg body weight). Mice were fasted overnight prior to re-feeding. Data represent means±SEM for 8 mice, *p<0.05, **p<0.01 and ***p<0.001 compared with saline alone.

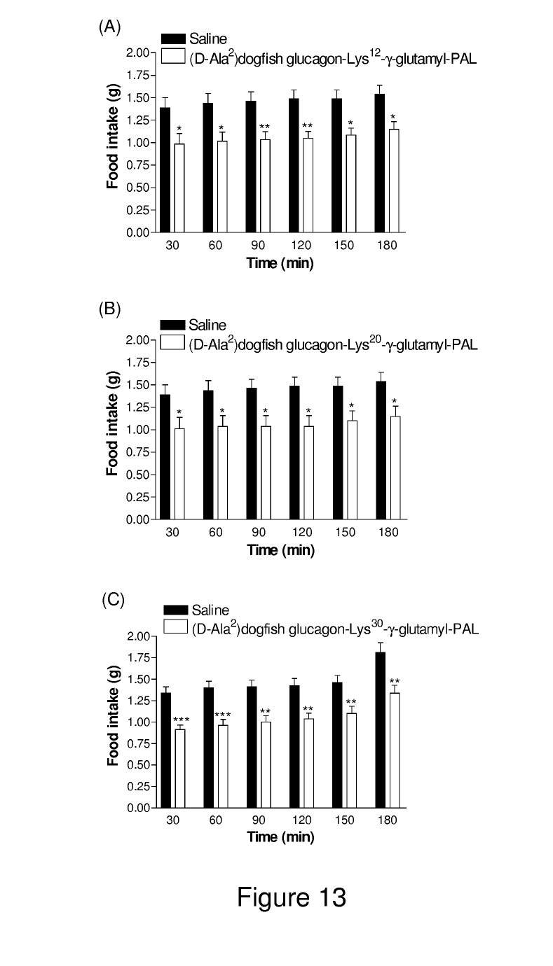

FIG. 13 shows the acute effects of (D-Ala²) dogfish glucagon-Lys¹²-γ-glutamyl-PAL (SEQ ID NO:18) (A), (D-Ala²) dogfish glucagon-Lys²⁰-γ-glutamyl-PAL (SEQ ID NO:17) (B) and (D-Ala²) dogfish glucagon-Lys³⁰-γ-glutamyl-PAL (SEQ ID NO:16) (C) on cumulative food intake in Swiss NIH mice. Food intake was measured in animals immediately following intraperitoneal injection with saline (0.9% (w/v), (D-Ala²) dogfish glucagon-Lys¹²-γ-glutamyl-PAL, (D-Ala²) dogfish glucagon-Lys²⁰-γ-glutamyl-PAL or (D-Ala²) dogfish glucagon-Lys³⁰-γ-glutamyl-PAL (each at 100 nmol/kg body weight). Mice were fasted overnight prior to re-feeding. Data represent means±SEM for 8 mice, *p<0.05, **p<0.01 and ***p<0.001 compared with saline alone.

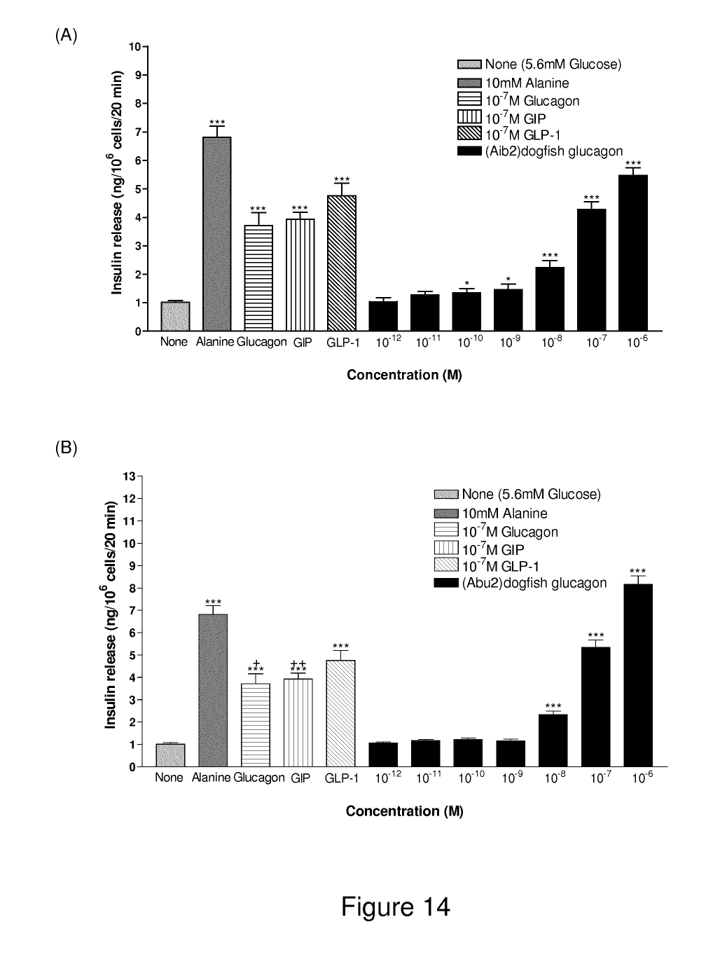

FIG. 14 shows the acute effects of increasing concentrations of (Aib²) dogfish glucagon (SEQ ID NO:8) (A) and (Abu²) dogfish glucagon (SEQ ID NO:9) (B) on insulin secretion from BRIN-BD11 clonal β cells in the presence of 5.6 mM glucose for 20 min and insulin release measured using radioimmunoassay. Values represent means±SEM (n=8) where *p<0.05 and ***p<0.001 compared with 5.6 mM glucose alone, +p<0.05 and ++p<0.01 compared to 10⁻⁷M (Aib²) dogfish glucagon (A) or 10⁻⁷M (Abu²) dogfish glucagon (B).

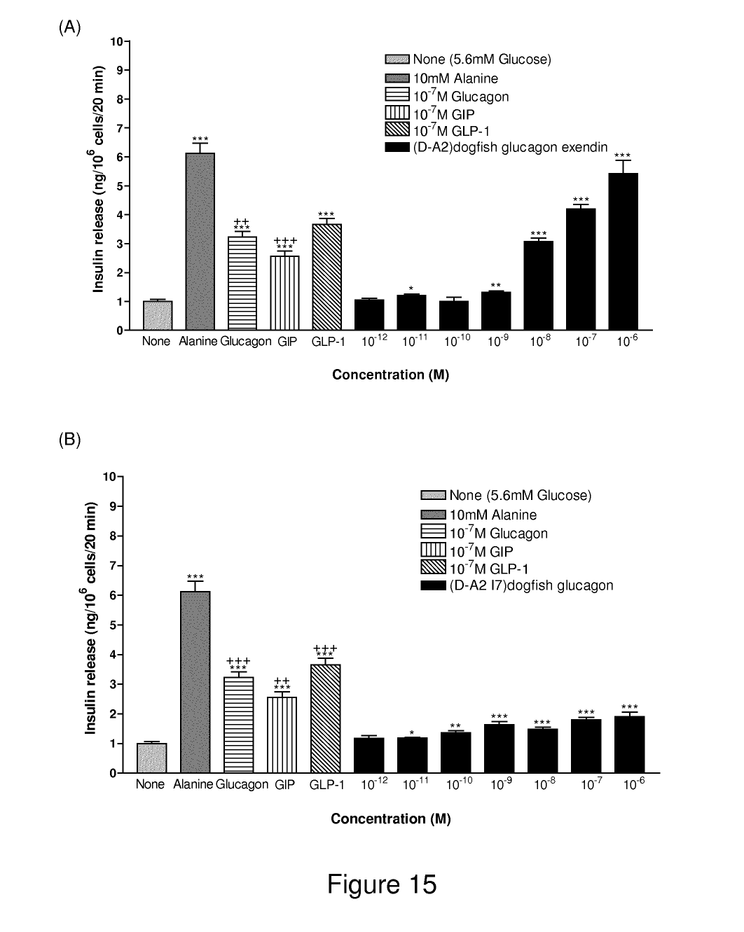

FIG. 15 shows the acute effects of increasing concentrations of (D-A²) dogfish glucagon exendin (SEQ ID NO:10) (A) and (D-A²I⁷) dogfish glucagon (SEQ ID NO:11) (B) on insulin secretion from BRIN-BD11 clonal β cells in the presence of 5.6 mM glucose for 20 min and insulin release measured using radioimmunoassay. Values represent means±SEM (n=8) where *p<0.05, **p<0.01 and ***p<0.001 compared with 5.6 mM glucose alone, ++p<0.01 and +++p<0.001 compared to 10⁻⁷M (D-A²) dogfish glucagon exendin (A) or 10⁻⁷M (D-A²I⁷) dogfish glucagon (B).

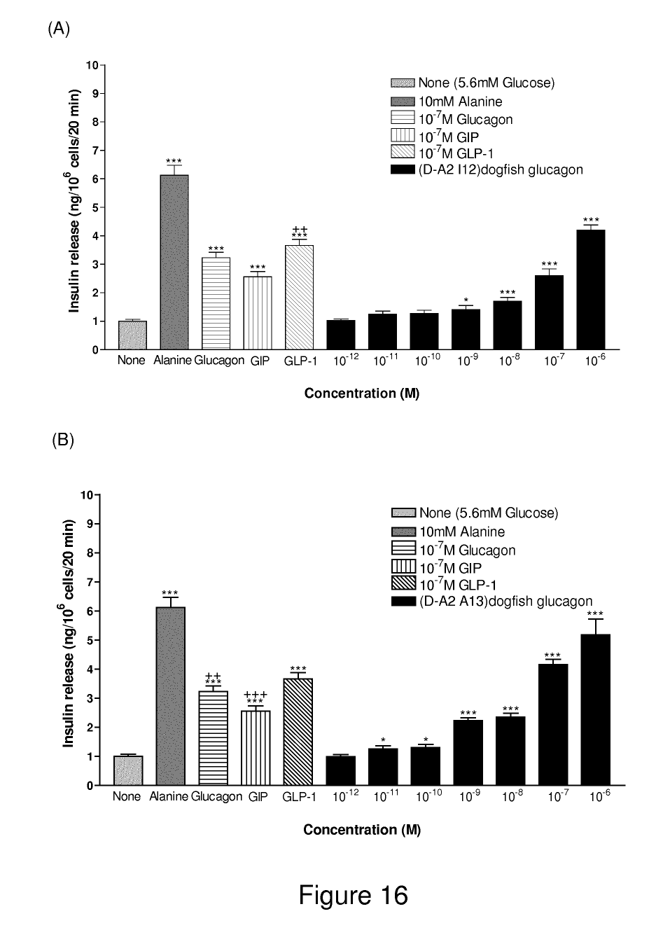

FIG. 16 shows the acute effects of increasing concentrations of (D-A²I¹²) dogfish) dogfish glucagon (SEQ ID NO:12) (A) and (D-A²A¹³) dogfish glucagon (SEQ ID NO:13) (B) on insulin secretion from BRIN-BD11 clonal β cells in the presence of 5.6 mM glucose for 20 min and insulin release measured using radioimmunoassay. Values represent means±SEM (n=8) where *p<0.05, **p<0.01 and ***p<0.001 compared with 5.6 mM glucose alone, ++p<0.01 and +++p<0.001 compared to 10⁻⁷ M (D-A²I¹²) dogfish glucagon (A) or 10⁻⁷M (D-A²A¹³) dogfish glucagon (B).

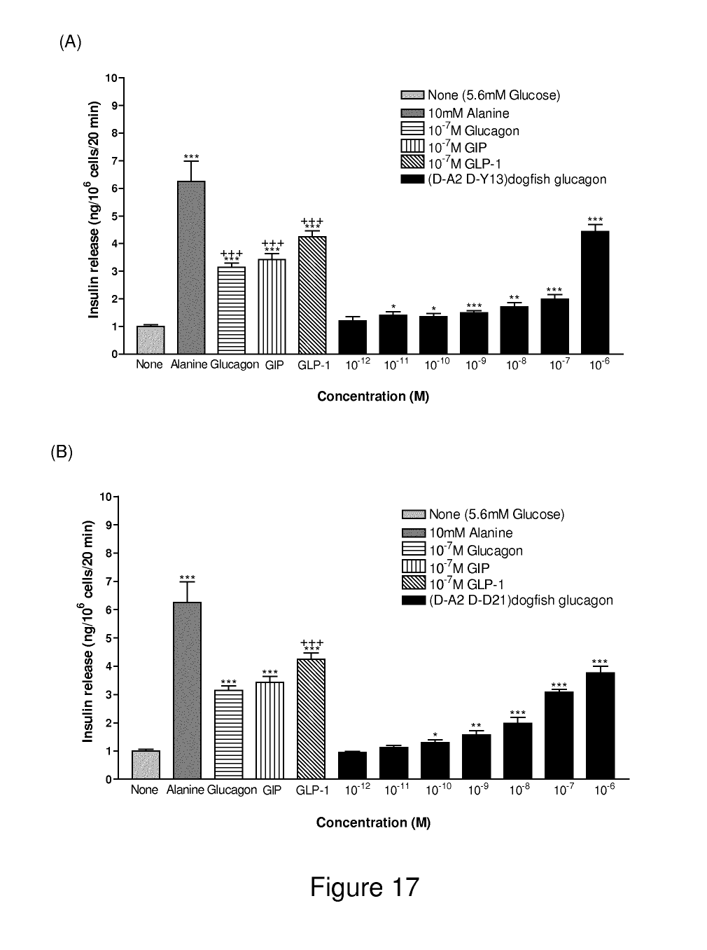

FIG. 17 shows the acute effects of increasing concentrations of (D-A²D-Y¹³) dogfish glucagon (SEQ ID NO:14) (A) and (D-A²D-D²¹) dogfish glucagon (SEQ ID NO:15) (B) on insulin secretion from BRIN-BD11 clonal β cells in the presence of 5.6 mM glucose for 20 min and insulin release measured using radioimmunoassay. Values represent means±SEM (n=8) where *p<0.05, **p<0.01 and ***p<0.001 compared with 5.6 mM glucose alone, +++p<0.001 compared to 10⁻⁷M (D-A² D-Y¹³) dogfish glucagon (A) or 10⁻⁷M (D-A² D-D²¹) dogfish glucagon (B).

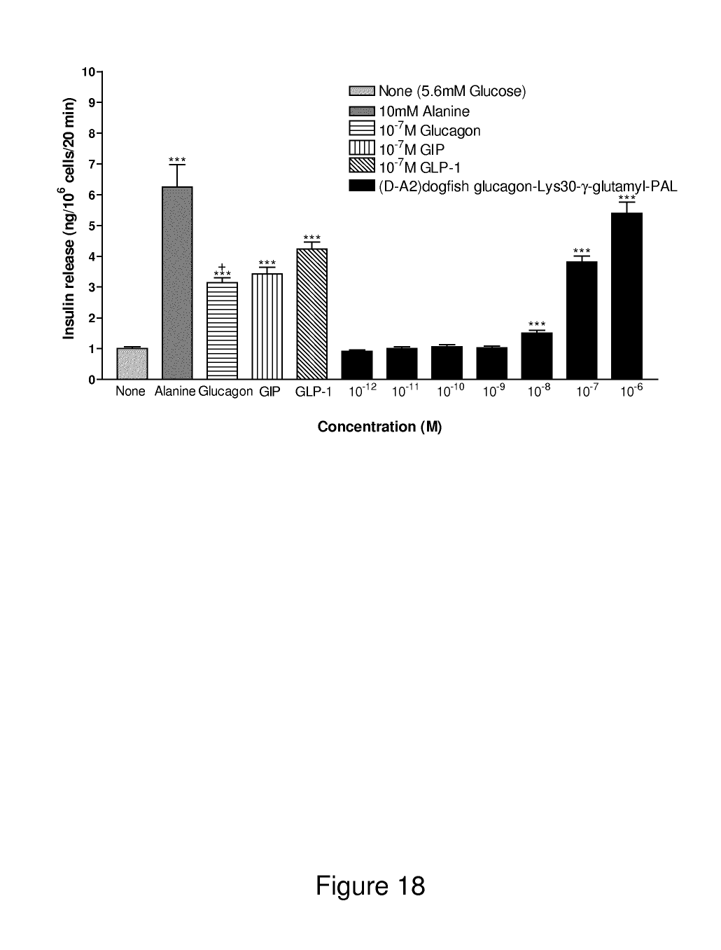

FIG. 18 shows the acute effects of increasing concentrations of (D-A²) dogfish glucagon-Lys³⁰-γ-glutamyl-PAL (SEQ ID NO:16) on insulin secretion from BRIN-BD11 clonal β cells in the presence of 5.6 mM glucose for 20 min and insulin release measured using radioimmunoassay. Values represent means±SEM (n=8) where ***p<0.001 compared with 5.6 mM glucose alone, +p<0.05 compared to 10⁻⁷M (D-A²) dogfish glucagon-Lys³⁰-γ-glutamyl-PAL.

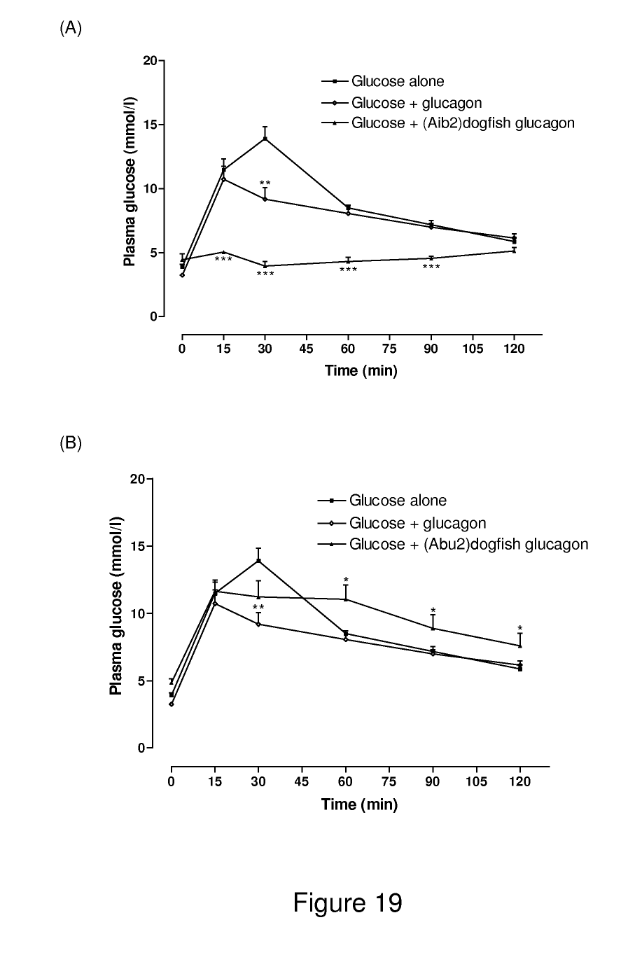

FIG. 19 shows the acute effects of (Aib²) dogfish glucagon (SEQ ID NO:8) (A) and (Abu²) dogfish glucagon (SEQ ID NO:9) (B) on glucose tolerance in Swiss NIH mice. Plasma glucose concentrations were measured prior to and after intraperitoneal administration of either (i) glucose alone (18 mmol/kg body weight) or (ii) glucose in combination with human glucagon (25 nmol/kg body weight), or (iii) glucose in combination with one of (Aib2) dogfish glucagon (A) or (Abu2) dogfish glucagon (B) (25 nmol/kg body weight). Data are expressed as means±SEM for 8 mice, *p<0.05, **p<0.01 and ***p<0.001 compared to glucose alone.

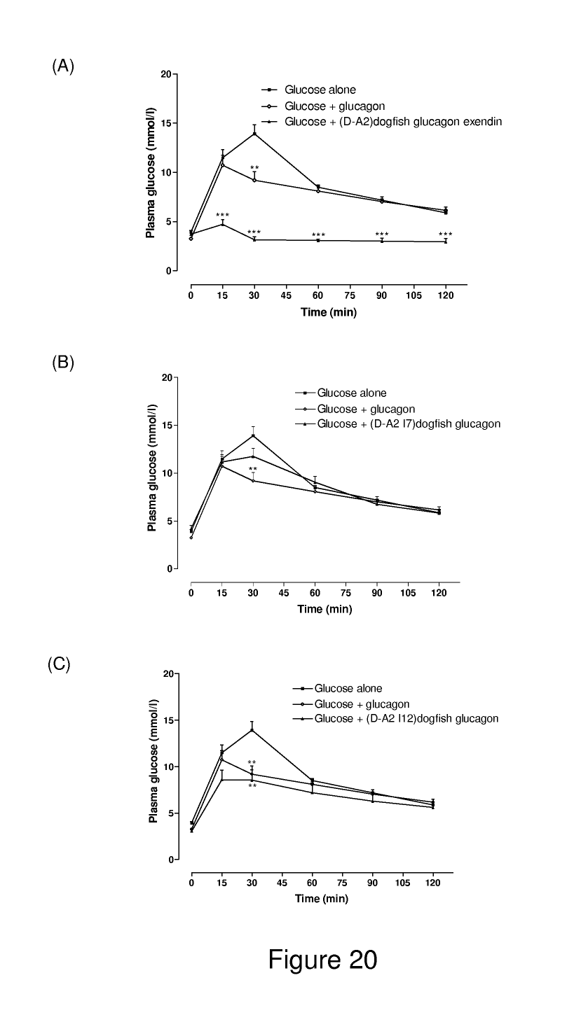

FIG. 20 shows the acute effects of (D-A²) dogfish glucagon exendin (SEQ ID NO:10) (A), (D-A² I⁷) dogfish glucagon (SEQ ID NO:11) (B), and (D-A² I¹²) dogfish glucagon (SEQ ID NO:12) (C) on glucose tolerance in Swiss NIH mice. Plasma glucose concentrations were measured prior to and after intraperitoneal administration of either (i) glucose alone (18 mmol/kg body weight) or (ii) glucose in combination with human glucagon (25 nmol/kg body weight), or (iii) glucose in combination with one of (D-A²) dogfish glucagon exendin (A), (D-A² I⁷) dogfish glucagon (B), or (D-A² I¹²) dogfish glucagon (C) (25 nmol/kg body weight). Data are expressed as means±SEM for 8 mice, **p<0.01 and ***p<0.001 compared to glucose alone.

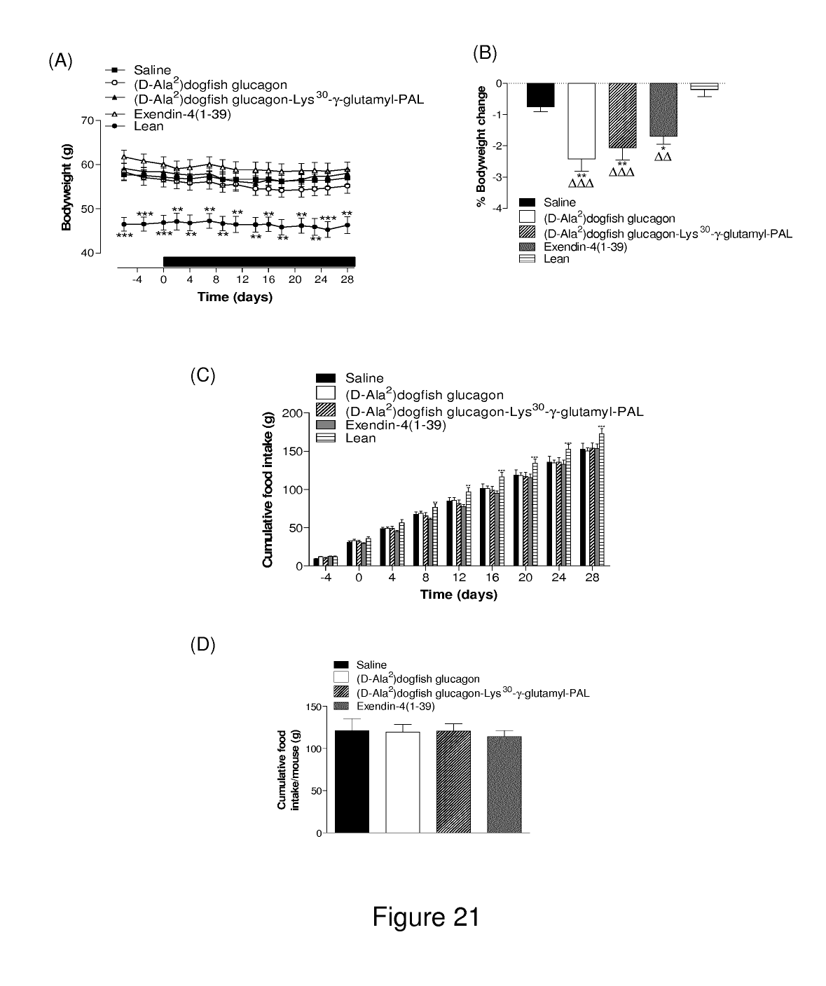

FIG. 21 shows the effect of twice daily administration of (D-Ala²) dogfish glucagon (SEQ ID NO: 7), (D-Ala²) dogfish glucagon-Lys³⁰-γ-glutamyl-PAL (SEQ ID NO: 16) and exendin-4(1-39), each at 25 nmol/kg bw) on body weight (A), bodyweight change (%) (B), daily food intake (C and D) in high-fat fed NIH Swiss mice. The black horizontal bar represents the treatment period within the 28-day study. Values represent mean±S.E.M. (n=8) where *p<0.05, **p<0.01 and ***p<0.001 compared with high-fat controls, ^{ΔΔ}p<0.01 and ^{ΔΔΔ}p<0.001 compared with lean controls.

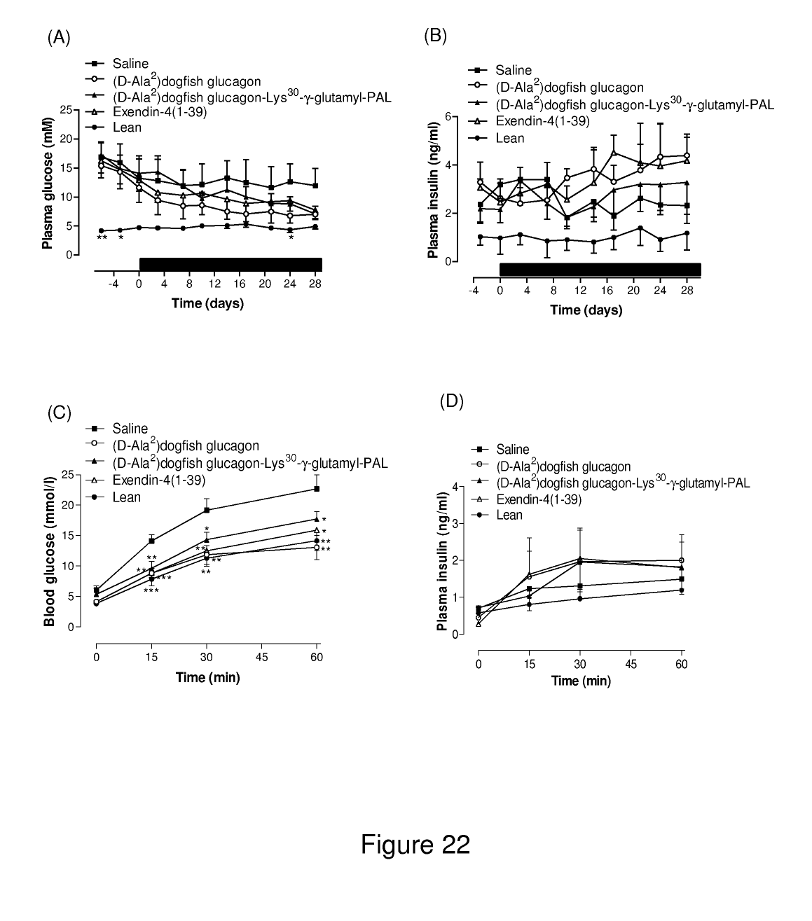

FIG. 22 shows the effect of twice daily administration of (D-Ala²) dogfish glucagon (SEQ ID NO: 7), (D-Ala²) dogfish glucagon-Lys³⁰-γ-glutamyl-PAL (SEQ ID NO: 16) and exendin-4(1-39), each at 25 nmol/kg bw, on non-fasting blood glucose (A) and plasma insulin (B) in high-fat fed NIH Swiss mice, and on blood glucose (C) and plasma insulin (D) in response to an i.p. glucose challenge in high-fat fed NIH Swiss mice.

(A) and (B)—The black horizontal bar represents the treatment period.

(C) and (D)—Tests were performed following 28 days of twice daily i.p. administration of saline ((0.9% w/v) NaCl), (D-Ala²) dogfish glucagon, (D-Ala²) dogfish glucagon-Lys³⁰-γ-glutamyl-PAL or exendin-4(1-39). Mice were fasted for 18 h previously. Blood glucose and insulin concentrations were measured prior to and after i.p. administration of glucose alone (18 mmol/kg bw).

(A) to (D)—Values represent the mean±S.E.M. (n=8) where *p<0.05, **p<0.01 and ***p<0.001 compared with high-fat controls.

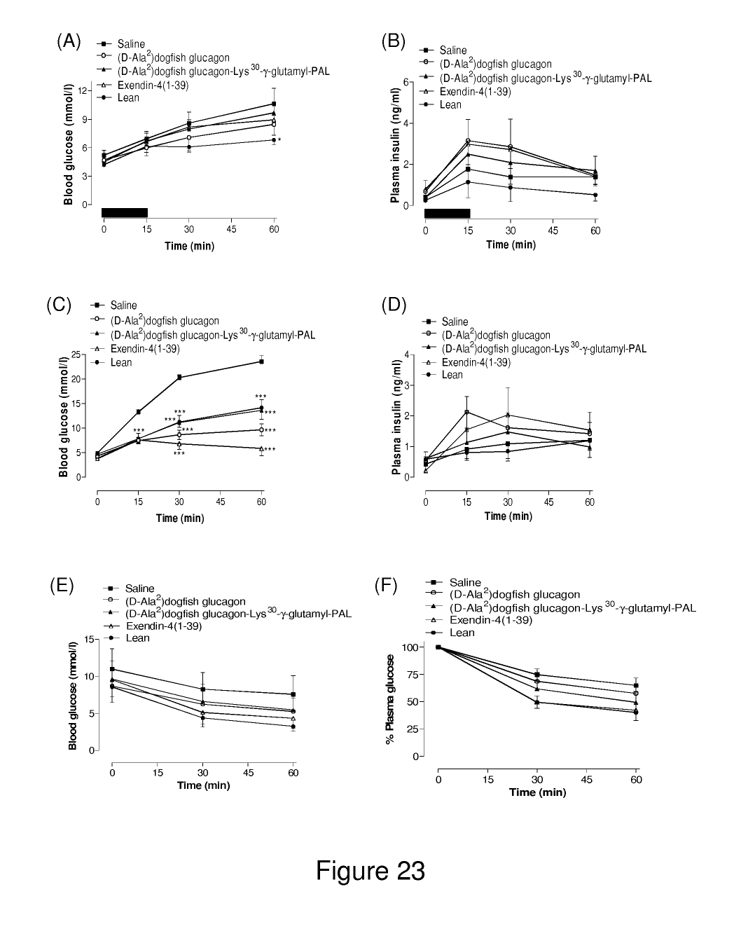

FIG. 23 shows the effect of twice daily administration of (D-Ala²) dogfish glucagon (SEQ ID NO: 7), (D-Ala²) dogfish glucagon-Lys³⁰-γ-glutamyl-PAL (SEQ ID NO: 16) and exendin-4(1-39) on blood glucose (A) and plasma insulin (B) in response to feeding, and on blood glucose (C) and plasma insulin (D) in response to peptide desensitisation, and on blood glucose (E) and plasma insulin (F) in response to insulin sensitivity in high-fat fed NIH Swiss mice. Tests were performed following 28 days of twice daily i.p. administration of saline ((0.9% w/v) NaCl), (D-Ala²) dogfish glucagon, (D-Ala²) dogfish glucagon-Lys³⁰-γ-glutamyl-PAL or exendin-4(1-39) (each at 25 nmol/kg bw).

(A) and (B)—Mice were fasted for 18 h previously and given free access to normal diet for 15 min. Blood glucose and insulin concentrations were measured at t=0, 15, 30, 60 min and time of feeding is represented by the black horizontal bar.

(C) and (D)—Mice were fasted for 18 h previous to experiment. Each peptide (at 25 nmol/kg bw) was administered in the presence of glucose (18 mmol/kg bw) at t=0 min after a baseline glucose reading was taken. Blood glucose and insulin concentrations were measured at t=0, 15, 30, 60 min.

(E) and (F)—Insulin (25 U/kg bw) was administered by intraperitoneal injection at t=0 min.

(A) to (F)—Values represent the mean±S.E.M. (n=8) where *p<0.05 and ***p<0.001 compared with high-fat controls.

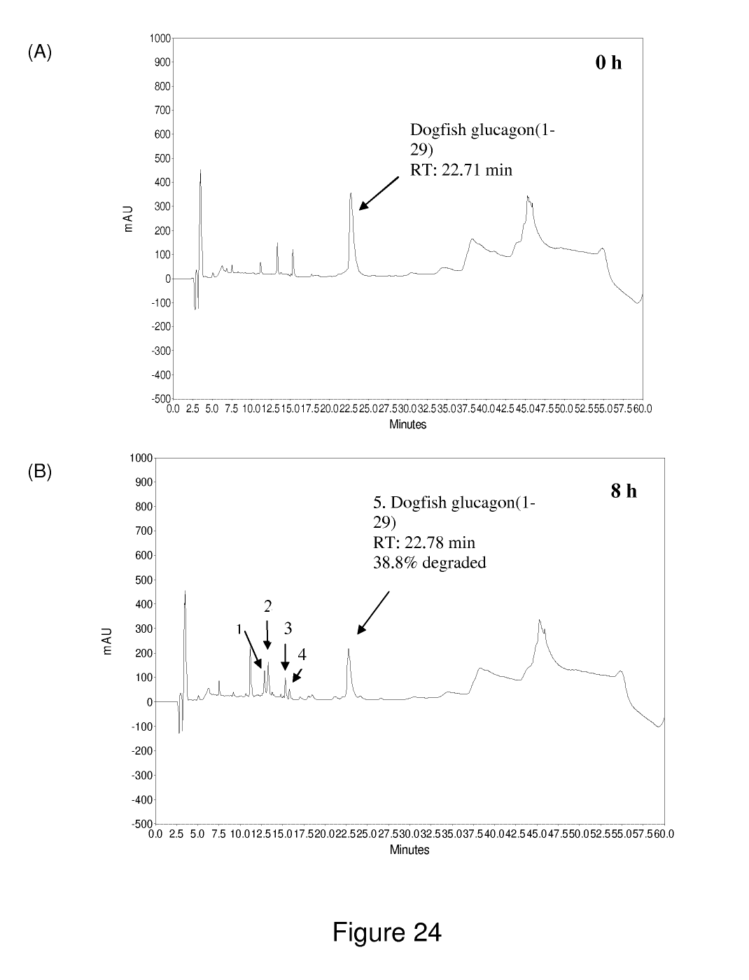

FIG. 24 shows the degradation of dogfish glucagon(1-29) (SEQ ID NO:5) by mouse plasma. Representative HPLC profiles obtained after incubation of dogfish glucagon(1-29) with mouse plasma for (A) 0 h and (B) 8 h. Dogfish glucagon(1-29) and its fragments were separated by RP-HPLC using a (250×4.6 mm) Jupiter C-8 analytical column. The percentage acetonitrile was raised from 0-40% over 10 min, to 60% over 40 min and to 70% over 5 min, monitoring the absorbance at 214 nm, at a flow rate of 1.0 ml/min.

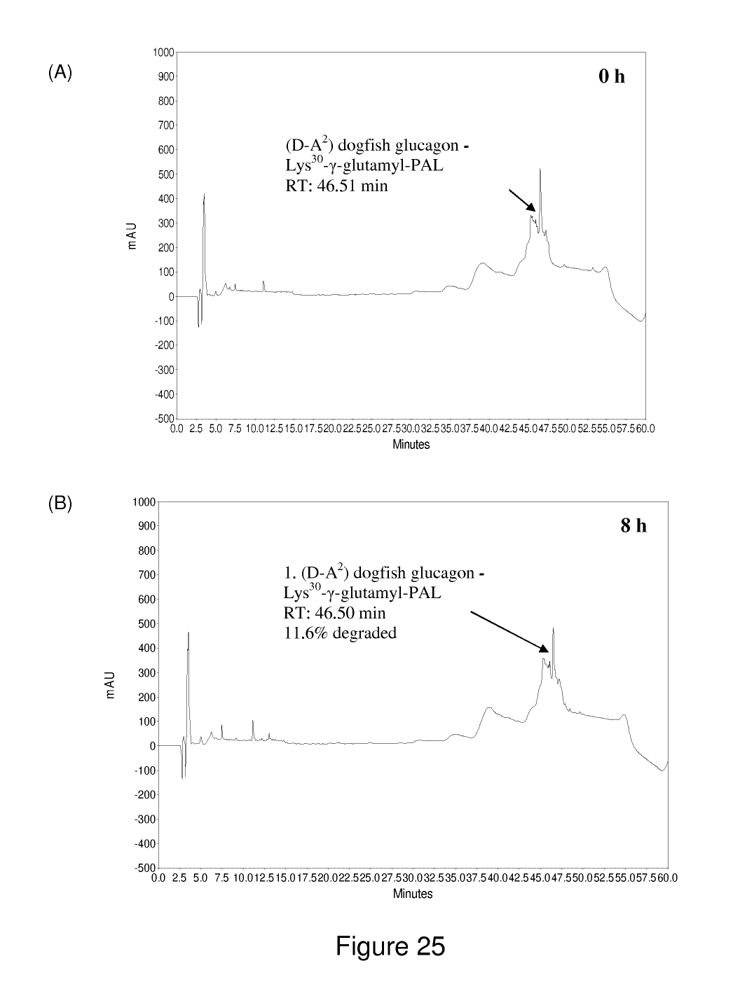

FIG. 25 shows the degradation of (D-A²) dogfish glucagon-Lys30-γ-glutamyl-PAL (SEQ ID NO: 16) by mouse plasma. Representative HPLC profiles obtained after incubation of (D-A²) dogfish glucagon-Lys30-γ-glutamyl-PAL with mouse plasma for (A) 0 h and (B) 8 h. (D-A²) dogfish glucagon-Lys³⁰-γ-glutamyl-PAL and its fragments were separated by RP-HPLC using a (250×4.6 mm) Jupiter C-8 analytical column. The percentage acetonitrile was raised from 0-40% over 10 min, to 60% over 40 min and to 70% over 5 min, monitoring the absorbance at 214 nm, at a flow rate of 1.0 ml/min.

Table 1 shows the native amino acid sequences of the human peptides glucagon, oxyntomodulin, gastric inhibitory polypeptide (GIP) and glucagon-like peptide 1 (GLP-1).

Table 2 shows the amino acid sequences of dogfish glucagon and analogues thereof. The residues that are underlined indicate the modifications that have been made as compared to the native dogfish glucagon sequence. “*” denotes a D chiral isomer of the amino acid immediately to the left of the symbol, rather than the naturally occurring L isomer, “K-γ-glutamyl-PAL” denotes a modified lysine residue, where a γ-glutamyl palmitate group is incorporated via an amide bond on the lysine side chain. “Aib” denotes 2-aminoisobutyric acid (aka 2-methylalanine). “Abu” denotes γ-aminobutyric acid.

### Tables

TABLE 1

| SEQ | | |
|---|---|---|
| ID NO | Name | Sequence |
| 1 | Human glucagon | HSQGTFTSDYSKYLDSRRAQDFVQWLMNT |
| 2 | Oxyntomodulin | HSQGTFTSDYSKYLDSRRAQDFVQWLMNT |
| | | KRNRNNIA |
| 3 | GIP (1-42) | YAEGTFISDYSIAMDKIHQQDFVNWLLAQ |
| | | KGKKNDWKHNITQ |
| 4 | GLP-1 (7-36) | HAEGTFTSDVSSYLEGQAAKEFIAWLVKGR |

TABLE 2

| SEQ | | |
|---|---|---|
| ID NO | Name | Sequence |
| 5 | Dogfish glucagon (1-29) | HSEGTFTSDYSKYMDNRRAKDFVQWLMNT |
| 6 | Y¹(D-A²) dogfish glucagon | YA*EGTFTSDYSKYMDNRRAKDFVQWLMNT |
| 7 | (D-A²) dogfish glucagon | HA*EGTFTSDYSKYMDNRRAKDFVQWLMNT |
| 8 | (Aib²) dogfish glucagon | HAibEGTFTSDYSKYMDNRRAKDFVQWLMNT |
| 9 | (Abu²) dogfish glucagon | HAbuEGTFTSDYSKYMDNRRAKDFVQWLMNT |
| 10 | (D-A²) dogfish glucagon | HA*EGTFTSDYSKYMDNRRAKDFVQWLMN |
| | exendin end | TPSSGAPPPSamide |
| 11 | (D-A²I⁷) dogfish glucagon | HA*EGTFISDYSKYMDNRRAKDFVQWLMNT |
| 12 | (D-A²I¹²) dogfish glucagon | HA*EGTFTSDYSIYMDNRRAKDFVQWLMNT |
| 13 | (D-A²A¹³) dogfish | HA*EGTFTSDYSKAMDNRRAKDFVQWLMNT |
| | glucagon | |
| 14 | (D-A²D-Y¹³) dogfish | HA*EGTFTSDYSKY*MDNRRAKDFVQWLMNT |
| | glucagon | |
| 15 | (D-A²D-D²¹) dogfish | HA*EGTFTSDYSKYMDNRRAKD*FVQWLMNT |
| | glucagon | |
| 16 | (D-A²) dogfish glucagon | HA*EGTFTSDYSKYMDNRRAKDFVQWLMN |
| | Lys³⁰-γ-Glutamyl-PAL | TK-γ-Glutamyl-PAL |
| 17 | (D-A²) dogfish glucagon | HA*EGTFTSDYSKYMDNRRA(K-γ-Glutamyl- |
| | Lys²⁰-γ-Glutamyl-PAL | PAL)DFVQWLMNT |
| 18 | (D-A²) dogfish glucagon- | HA*EGTFTSDYS(K-γ-Glutamyl- |
| | Lys¹²-γ-Glutamyl-PAL | PAL)YMDNRRA KDFVQWLMNT |

### EXAMPLES

### Example 1

### Actions of Dogfish Glucagon and Dogfish Glucagon Analogues on In Vitro Insulin Secretion

The glucose-mediated insulin-secreting effects of dogfish glucagon (SEQ ID NO:5), (Tyr¹)(D-Ala²) dogfish glucagon (SEQ ID NO:6) and (D-Ala²) dogfish glucagon (SEQ ID NO:7) were assessed against human glucagon (SEQ ID NO:1) in order to determine their potential as therapeutic agents. The pancreatic BRIN-BD11 cell line was used to perform in vitro studies.

In a further study, the GLP-1R antagonist Exendin-4(9-39), the GIPR antagonist (Pro³) GIP and the GCGR antagonist (desHis¹)(Pro³) glucagon were used in competition experiments in order to determine which receptors the dogfish glucagon analogue (D-Ala²) dogfish glucagon (SEQ ID NO:7) may be interacting with.

### Methods

BRIN-BD11 cells (ECACC accession number 100330033) were cultured in RPMI 1640 tissue culture medium containing 10% (v/v) foetal calf serum, 1% (v/v) antibiotics (100 U/ml of penicillin and 0.1 mg/ml of streptomycin). BRIN-BD11 cells were originally derived by means of electrofusion of a New England Deaconess Hospital (NEDH) rat pancreatic β cell line with an RINm5F cell line in order to produce an immortal, glucose sensitive, insulin secreting cell line (McClenaghan et. al., 1996). All cells were maintained in sterile tissue culture flasks (Corning Glass Works, Corning, N.Y., USA) at 37° C. in an atmosphere of 5% CO₂ and 95% air, using an LEEC incubator (Laboratory Technical Engineering, Nottingham, UK).

BRIN-BD11 cells were seeded into 24-well plates at a density of 1×10⁵ cells and allowed to attach to the wells overnight in an incubator. Before acute studies of insulin were carried out, the cells underwent a 40 minute preincubation period at 37° C. with 1.0 ml of Krebs Ringer bicarbonate buffer (115 mM NaCl, 4.7 mM KCl, 1.28 mM CaCl₂.2H₂O, 1.2 mM KH₂PO₄, 1.2 mM MgSO₄.7H₂O, 10 mM NaHCO₃, 5 g/l bovine serum albumin (BSA), pH 7.4) supplemented with 1.1 mM glucose. Acute test incubations were performed at 37° C. in the presence of 5.6 mM glucose with concentration range of 10⁻¹² to 10⁻⁶ M and each analogue alone as well as in combination with (Pro³) GIP, (desHis¹)(Pro⁴) glucagon and Exendin-4(9-39). The incubations were performed for 20 minute at 37° C., after which the buffer solution was removed and 2×200 μl aliquots stored at −20° C. until measurement of insulin levels by radioimmunoassay.

### Results

As shown in FIGS. 2 and 3, glucagon, dogfish glucagon, (Tyr¹)(D-Ala²) dogfish glucagon and (D-Ala²) dogfish glucagon all significantly stimulated insulin secretion; with (D-Ala²) dogfish glucagon being the most potent of all the peptides. Increasing concentrations (10⁻¹²M-10⁻⁶M) of the antagonists (Exendin-4(9-39), (Pro³) GIP and (desHis¹)(Pro⁴) glucagon) alone had no effect on insulin secretion. Increasing concentrations of (Pro³) GIP and (desHis¹)(Pro⁴) glucagon had no effect in the presence of a fixed concentration (10⁻⁷M) of (D-Ala²) dogfish glucagon (FIGS. 5 and 6), suggesting that (D-Ala²) dogfish glucagon does not act through the GIP receptor nor the glucagon receptor. However, Exendin-4(9-39) appears to have a dose-dependent effect on (D-Ala²) dogfish glucagon, with increasing concentrations of the antagonist lowering the insulin-secreting capability of the agonist (FIG. 4), suggesting a role for the GLP-1 receptor and the insulin-secreting effects of (D-Ala²) dogfish glucagon.

### Example 2

### In Vivo Analysis of the Effects of Dogfish Glucagon and Dogfish Glucagon Analogues on Insulin Secretion and Glucose Homeostasis

In vivo animal studies in Swiss NIH mice were used to determine the effect of the dogfish glucagon and dogfish glucagon analogues on insulin secretion and glucose homeostasis. Acute glucose tolerance tests were carried out using dogfish glucagon (SEQ ID NO:5), (D-Ala²) dogfish glucagon (SEQ ID NO:7), (Tyr⁻¹)(D-Ala²) dogfish glucagon (SEQ ID NO:6), (D-Ala²) dogfish glucagon-Lys²⁰-γ-glutamyl-PAL (SEQ ID NO:17), (D-Ala²) dogfish glucagon-Lys¹²-γ-glutamyl-PAL (SEQ ID NO:18) and (D-Ala²) dogfish glucagon-Lys³⁰-γ-glutamyl-PAL (SEQ ID NO:16), and radioimmunoassay was used to determine insulin concentrations.

Longer terms studies (4 hours post-peptide administration) were also performed for (D-Ala²) dogfish glucagon-Lys¹²-γ-glutamyl-PAL (SEQ ID NO:18), (D-Ala²) dogfish glucagon-Lys²⁰-γ-glutamyl-PAL (SEQ ID NO:17) and (D-Ala²) dogfish glucagon-Lys³⁰-γ-glutamyl-PAL (SEQ ID NO:16).

### Methods

Young male Swiss NIH mice (6-8 weeks old; Harlan Ltd., Blackthorn, UK) were age-matched and housed individually in an air conditioned room at 22±2° C. with a 12 h light: 12 h dark cycle (08:00-20:00 h). Animals had free access to drinking water and a standard chow diet.

All blood samples were collected from the tail vein of conscious mice into fluoride coated microcentrifuge tubes (Sarstedt, Numbrecht, Germany) at the time points indicated in the FIGS. 7 to 9. Once collected, the samples were immediately centrifuged using a Beckman microcentrifuge (Beckman Instruments, Galway, Ireland) for 30 s at 13,000×g. The separated plasma was aliquoted into 500 μl Eppendorf tubes and stored at −20° C. prior to biochemical analysis of the metabolic parameters.

A pre-injection baseline blood glucose reading was taken using an Ascencia Contour glucose meter and analysis strips (Bayer Healthcare, UK). Glucose concentrations were subsequently measured using an Ascencia Contour glucose meter following intra-peritoneal administration of saline ((0.9% w/v) NaCl), glucose alone (18 mmol/kg body weight), peptide alone (each at 25 nmol/kg body weight) or a peptide (25 nmol/kg body weight) in the presence of glucose at 15, 30 and 60 minutes post-injection. Blood samples were also collected from the tail vein of conscious mice at the various time points into fluoride coated microcentrifuge tubes (Sarstedt, Numbrecht, Germany) for determination of plasma insulin. The collected blood samples were immediately centrifuged using a Beckman microcentrifuge (Beckman Instruments, Galway, Ireland) for 30 seconds at 13,000×g. The resultant plasma was aliquoted into 500 μl Eppendorf tubes and stored at −20° C. prior to plasma insulin analysis.

Plasma insulin levels were determined by a modified dextran-coated charcoal radioimmunoassay as described in Flatt & Bailey (Diabetologia, 1981, 20, pp 573-7).

### Results

FIG. 7 shows that (Tyr¹)(D-Ala²) dogfish glucagon was not able to reduce blood glucose compared to glucose alone whilst dogfish glucagon and (D-Ala²) dogfish glucagon both significantly reduced blood glucose compared to glucose alone. Varying effects were observed with the fatty-acid derivatives of (D-Ala²) dogfish glucagon; immediately following injection with (D-Ala2) dogfish glucagon-Lys³⁰-γ-glutamyl-PAL, glucose levels decreased more significantly compared to (D-Ala²) dogfish glucagon-Lys¹²-γ-glutamyl-PAL and (D-Ala²) dogfish glucagon-Lys²⁰-γ-glutamyl-PAL (FIG. 8). However, after a 4 hour delayed GTT the most significant effect was observed with (D-Ala²) dogfish glucagon-Lys²⁰-γ-glutamyl-PAL when compared with the other fatty-acid derivatives of (D-Ala²) dogfish glucagon (FIG. 9).

### Example 3

### Receptor Agonism Analysis In Vivo of Dogfish Glucagon (SEQ ID NO:5) and (D-ala²) Dogfish Glucagon (SEQ ID NO:7) Using C57 Control Mice, GLP-1R Knockout Mice and GIPR-KO Mice

Acute in vivo animal studies in C57 control, GIPR-KO and GLP-1R-KO mice were performed to generate a better understanding of receptor binding and activation.

### Methods

Young female C57bl6, GIPr-KO and GLP-1r-KO mice (age) were age-matched and housed in groups of 7 or 8 in an air-conditioned room at 22±2° C. with a 12 h light: 12 h dark cycle (08:00-20:00 h). Animals had free access to drinking water and a standard chow diet.

Peptide and glucose administration, blood glucose level determination and plasma insulin level determination were carried out as described in Example 2.

### Results

Dogfish glucagon significantly lowered glucose in both C57 and GLP-1r-KO, whereas it had no glucose-lowering effects in GIPR-KO mice compared to glucose alone (FIG. 10), suggesting a role for the GIP receptor and the insulin-secreting effects of dogfish glucagon. An effect was observed in C57 and GIPR-KO with (D-Ala²) dogfish glucagon, however no significant difference was observed compared with glucose alone in GLP-1R-KO mice (FIG. 11) suggesting a role for the GLP-1 receptor and the insulin-secreting effects of (D-Ala²) dogfish glucagon.

### Example 4

### Acute In Vivo Food Intake Studies with Dogfish Glucagon (SEQ ID NO:5) and Dogfish Glucagon Analogues

Acute in vivo food intake studies were carried out using dogfish glucagon and selected dogfish glucagon analogues at a concentration of 100 nmol/kg body weight in order to determine food inhibition effects in Swiss NIH mice over a 3 hour period.

### Methods

Mice were given an i.p. administration of saline or peptide (each at 25 nmol/kg bw) and then allowed free access to a pellet of standard chow over a 3 hour period. Mice were fasted overnight prior to re-feeding. Food intake was measured immediately following injection at 30 min intervals up to 180 min as indicated in the FIG. 12 and FIG. 13.

### Results

All of the peptides tested significantly inhibited food intake at a concentration of 100 nmol/kg body weight (FIGS. 12 and 13). The most significant effects were seen after the administration of (Tyr¹)(D-Ala²) dogfish glucagon (SEQ ID NO:6)

### Example 5

### Actions of further Dogfish Glucagon Analogues on In Vitro Insulin Secretion

In Example 1, the glucose-mediated insulin-secreting effects of dogfish glucagon (SEQ ID NO:5), (Tyr¹)(D-Ala²) dogfish glucagon (SEQ ID NO:6) and (D-Ala²) dogfish glucagon (SEQ ID NO:7) were assessed against human glucagon (SEQ ID NO:1) in order to determine their potential as therapeutic agents. The pancreatic BRIN-BD11 cell line was used to perform in vitro studies.

Here, the glucose-mediated insulin-secreting effects of (Aib²) dogfish glucagon (SEQ ID NO:8) (A), (Abu²) dogfish glucagon (SEQ ID NO:9), (D-A²) dogfish glucagon exendin (SEQ ID NO:10), (D-A²I⁷) dogfish glucagon (SEQ ID NO:11), (D-A²I¹²) dogfish glucagon (SEQ ID NO:12), (D-A²A¹³) dogfish glucagon (SEQ ID NO:13), (D-A² D-Y¹³) dogfish glucagon (SEQ ID NO:14) (A), (D-A² D-D²¹) dogfish glucagon (SEQ ID NO:15) and (D-A²) dogfish glucagon-Lys³⁰-γ-glutamyl-PAL (SEQ ID NO:16) were assessed against human glucagon (SEQ ID NO:1) in order to determine their potential as therapeutic agents. The pancreatic BRIN-BD11 cell line was again used to perform in vitro studies.

### Methods

The methodology was as described for Example 1.

### Results

As shown in FIGS. 14 to 18, all of the dogfish glucagon analogues tested significantly stimulated insulin secretion compared to 5.6 mM glucose alone. In particular, analogues (Aib²) dogfish glucagon (SEQ ID NO:8), (Abu²) dogfish glucagon (SEQ ID NO:9), (D-A²) dogfish glucagon exendin (SEQ ID NO:10) were among the most potent analogues tested in this Example.

### Example 6

### In Vivo Analysis of the Effects of further Dogfish Glucagon Analogues on Insulin Secretion and Glucose Homeostasis

In Example 2, the in vivo effects of dogfish glucagon (SEQ ID NO:5), (D-Ala²) dogfish glucagon (SEQ ID NO:7), (Tyr¹)(D-Ala²) dogfish glucagon (SEQ ID NO:6), (D-Ala²) dogfish glucagon-Lys²⁰-γ-glutamyl-PAL (SEQ ID NO:17), (D-Ala²) dogfish glucagon-Lys¹²-γ-glutamyl-PAL (SEQ ID NO:18) and (D-Ala²) dogfish glucagon-Lys³⁰-γ-glutamyl-PAL (SEQ ID NO:16) on insulin secretion and glucose homeostasis were assessed in Swiss NIH mice.

Here, the in vivo effects of further dogfish glucagon analogues on glucose homeostasis were assessed in Swiss NIH mice. Thus, acute glucose tolerance tests were carried out using (Aib²) dogfish glucagon (SEQ ID NO:8), (Abu²) dogfish glucagon (SEQ ID NO:9), (D-A²) dogfish glucagon exendin (SEQ ID NO:10), (D-A² I⁷) dogfish glucagon (SEQ ID NO:11) and (D-A² I¹²) dogfish glucagon (SEQ ID NO:12).

### Methods

Young male Swiss NIH mice (6-8 weeks old; Harlan Ltd., Blackthorn, UK) were age-matched and housed individually in an air conditioned room at 22±2° C. with a 12 h light: 12 h dark cycle (08:00-20:00 h). Animals had free access to drinking water and a standard chow diet.

All blood samples were collected from the tail vein of conscious mice into fluoride coated microcentrifuge tubes (Sarstedt, Numbrecht, Germany) at the time points indicated in the FIGS. 19 and 20. Once collected, the samples were immediately centrifuged using a Beckman microcentrifuge (Beckman Instruments, Galway, Ireland) for 30 s at 13,000×g. The separated plasma was aliquoted into 500 μl Eppendorf tubes and stored at −20° C. prior to biochemical analysis of the metabolic parameters.

A pre-injection baseline blood glucose reading was taken using an Ascencia Contour glucose meter and analysis strips (Bayer Healthcare, UK). Glucose concentrations were subsequently measured using an Ascencia Contour glucose meter following intra-peritoneal administration of glucose alone (18 mmol/kg body weight), glucose in combination with human glucagon or with the analogue of interest (each at 25 nmol/kg body weight) at 15, 30, 60, 90 and 120 minutes post-injection.

### Results

FIGS. 19 and 20 show that all of (Aib²) dogfish glucagon (SEQ ID NO:8), (Abu²) dogfish glucagon (SEQ ID NO:9), (D-A²) dogfish glucagon exendin (SEQ ID NO:10), (D-A² I⁷) dogfish glucagon (SEQ ID NO:11) and (D-A² I¹²) dogfish glucagon (SEQ ID NO:12) significantly reduced blood glucose at various time points compared to glucose alone. In particular, these analogues displayed the ability to decrease circulating glucose concentrations significantly within 30 minutes following their injection. The most significant effects at early time points (15 min) were observed with (Aib²) dogfish glucagon (SEQ ID NO:8) and (D-A²) dogfish glucagon exendin (SEQ ID NO:10), and these analogues were even shown to reduce plasma glucose levels at later time points as compared to the administration of glucose alone or glucose in combination with human glucagon.

### Example 7

### In Vivo Analysis of the Effects of (D-A2) Dogfish Glucagon (SEQ ID NO: 7) and (D-A2) Dogfish Glucagon-Lys30-γ-Glutamyl-PAL (SEQ ID NO:16) on Metabolic Control in a Dietary Induced Model of Obesity-Diabetes

A chronic 28 day study was performed to established the efficacy of chronic administration of the GLP-1 mimetic exendin-4 in comparison to novel treatments in the form of (D-Ala²) dogfish glucagon (SEQ ID NO:7) and (D-Ala²) dogfish glucagon-Lys³⁰-γ-glutamyl-PAL (SEQ ID NO:16) on metabolic control in a dietary induced model of obesity-diabetes.

### Methods

Animals—NIH Swiss mice (Harlan UK Ltd., Blackthorne, UK) were derived from a nucleus colony obtained from the National Institute of Health, Bethesda, Md. One group of mice were maintained on a standard rodent diet (lean diet) (10% fat, 30% protein, 60% carbohydrate; percent of total energy 12.99 kJ/g, Trouw Nutrition, Cheshire, UK) and used as a model of normal glycaemia. Normal mice with similar bodyweight and blood glucose concentrations were selected as saline-treated (placebo) controls for this study (n=8).

NIH Swiss mice were also maintained on a high fat diet (45% fat, 20% protein, 35% carbohydrate; percent of total energy 26.15 kJ/g; Special Diet Services, Essex, UK) from 8 weeks of age for 150 days to produce a model of diet induced obesity-diabetes. High-fat fed mice exhibiting increased body weight and elevated non-fasting blood glucose were selected for studies (n=8). Mice were housed in an air-conditioned room maintained at 22±2° C. with a 12 h light: 12 h dark cycle (08:00-20:00 h), were single caged, and grouped according to body weight and non-fasted blood glucose. Drinking water and standard rodent maintenance diet or high-fat diet, were freely available. All animal studies were performed in accordance with the UK Animals (Scientific Procedures) Act 1986.

Treatments—Normal control NIH Swiss and high-fat fed NIH Swiss were grouped and received twice daily i.p. injections of saline vehicle (0.9% NaCl (w/v)) at 09:00 and 18:00 h over a 7 day run-in period. Following the run-in period normal control (n=8) and high-fat fed mice (n=8) received twice daily (09:00 and 18:00 h) i.p. administration of saline vehicle (0.9% NaCl (w/v)) for 28 days. Additional groups of high-fat fed mice (n=8) received twice daily i.p. injections of (D-Ala²) dogfish glucagon, (D-Ala²) dogfish glucagon-Lys³⁰-γ-glutamyl-PAL or exendin(1-39) (each at 25 nmol/kg bw) over a 28 day treatment period.

Measurement of metabolic effects—Food intake, body weight, blood glucose and plasma insulin were monitored at intervals of 2-3 days throughout the run-in and 28 day treatment. Blood samples for glucose and plasma insulin measurement were collected at intervals throughout the study from the cut tail vein of conscious mice. Blood glucose was measured using an Ascensia Contour meter and blood was collected for plasma insulin analysis by radioimmunoassay.

Following the 28 day treatment period glucose tolerance (18 nmol/kg bw; i.p.), feeding, peptide desensitisation (25 nmol/kg bw) and insulin sensitivity (25 U/kg bw) tests were performed as follows. Mice were assessed for desensitisation to peptide analogues. Animals were fasted 18 h prior to administration of glucose alone (18 mmol/kg bw) or glucose in combination with the peptide analogues (each at 25 nmol/kg bw). Blood samples were collected from the tail vein of conscious mice for blood glucose and plasma insulin analysis immediately prior (t=0) and at 15, 30, and 60 min post injection. Blood glucose was measured immediately using a handheld Acensia Contour meter (Bayer Healthcare, UK). Blood samples for insulin analysis were collected into fluoride coated microcentifuge tubes (Sarstedt, Numbrecht, Germany) and kept chilled on ice. Collected blood samples were centrifuged and stored at −20° C. prior to insulin analysis by radioimmunassay.

For insulin sensitivity tests blood glucose was measured from the tail vein of non-fasted conscious mice using a handheld Acensia Contour glucose meter (Bayer Healthcare, UK). Blood glucose was measured immediately prior to (t=0) and following intraperitoneal administration of bovine insulin (25 U/kg bodyweight) at 30 and 60 min post injection.

Fat mass, bone mineral content (BMC) and bone mineral density (BMD) were also assessed using the PIXImus DEXA scanner.

Terminal analysis—At termination, pancreatic tissues were excised for analysis of pancreatic insulin content. Thawed tissue was rinsed in cold PBS before being weighed and transferred to a pestle and mortar, where it was homogenised in liquid nitrogen. The contents were carefully transferred to a beaker where 40 ml of ice-cold acid ethanol (1.5% (v/v) HCl, 75% (v/v) ethanol, 23.5% (v/v) H₂0) was added per gram of tissue used. Insulin was extracted from the homogenised tissue with rocking in a refrigerated room over 3 h, spun at 50×g for 5 min and the resulting supernatant transferred to a fresh tube. Samples were then diluted to a range of concentrations (1:10, 1:100, 1:500, and 1:1000) using stock RIA buffer. Diluted samples (200 μl) were transferred to LP3 tubes (Sarstedt, Germany) for insulin radioimmunoassay.

Blood was also taken for measurement of lipid profiles by an ILab650 clinical analyser (Instrumentation Laboratory, Warrington, UK). This included assessment of total triglycerides, total cholesterol, high-density lipoproteins and low-density lipoproteins. Reagents for triglycerides analysis were obtained from Instrumentation Laboratory (Warrington, UK) and reagents for LDL cholersterol were obtained from Randox (Randox, Co. Antrim, UK).

Statistical analysis—Data was expressed as mean±S.E.M. and values compared using a one-way ANOVA, followed by a Student Newman-Keuls post-hoc test. Groups of data were considered significantly different if p<0.05. The area under the curve (AUC) was also calculated by using GraphPad PRISM (CA, USA) Version 3.0.

### Results

A progressive decline in non-fasted blood glucose is evident with all groups of mice treated with twice daily administration of (D-Ala²) dogfish glucagon, (D-Ala²) dogfish glucagon-Lys³⁰-γ-glutamyl-PAL or exendin(1-39) (FIGS. 22A and B). In addition, by the end of the study all high-fat fed peptide treated mice had similar blood glucose levels when compared with lean control mice (FIG. 22A). Also, plasma insulin concentrations progressively increased with all treatment groups over the 28 day treatment period when compared with high-fat controls (FIG. 22B). A significant decrease in % bodyweight change was noted with all treatment groups (p<0.05 to p<0.01; FIG. 21B).

Administration of (D-Ala²) dogfish glucagon, (D-Ala²) dogfish glucagon-Lys³⁰-γ-glutamyl-PAL or exendin(1-39) for 28 days significantly improved the glycaemic response at 15, 30 and 60 min post i.p. glucose load compared to high-fat fed control mice (p<0.05 to p<0.001; FIG. 22C). In addition, the overall glycaemic excursion was also significantly lowered in (D-Ala²) dogfish glucagon peptide treated mice compared to high-fat saline controls (p<0.05; FIG. 22C). (D-Ala²) dogfish glucagon significantly increased the overall plasma insulin release post-glucose load compared with high-fat saline controls (FIG. 22D).

Chronic administration of (D-Ala²) dogfish glucagon or exendin(1-39) for 28 days resulted in a progressive decline in blood glucose concentrations over the 60 min period when compared with high-fat fed saline controls (FIG. 23A). Plasma insulin levels increased over time with both (D-Ala²) dogfish glucagon and exendin-4 (FIG. 23B).

High-fat fed mice were administered (D-Ala²) dogfish glucagon, (D-Ala²) dogfish glucagon-Lys³⁰-γ-glutamyl-PAL or exendin(1-39) for 28 days, desensitisation for each of the peptides was then assessed. When given in combination with glucose, all peptides significantly inhibited the glucose-mediated rise in blood glucose at 15, 30 and 60 min post-peptide administration (p<0.001; FIG. 23C). Peptides also effectively inhibited the overall rise in blood glucose when compared to high-fat fed saline controls. Overall basal plasma insulin levels were significantly increased with (D-Ala²) dogfish glucagon compared with saline controls (FIG. 23D).

Sensitivity to the glucose-lowering effects of insulin at the time-points recorded and the overall insulin levels were determined by AUC analysis. AUC analysis showed a significant increase in insulin sensitivity with exendin-4 treated mice compared with high-fat controls (p<0.01; FIG. 23F).

### Example 8

### Stability Studies with Dogfish Glucagon (SEQ ID NO:5) and Dogfish Glucagon Analogues

Stability studies were carried out using dogfish glucagon and selected dogfish glucagon analogues.

### Methods

Human glucagon (SEQ ID NO:1), dogfish glucanon (SEQ ID NO: 5) and the analogues thereof of SEQ ID NOs: 7, 8, 9, 10, 11, 12, 13, 14, 15 and 16 were incubated with mouse plasma for 8 hours. The samples were then separated on RP-HPLC and the peak height compared to the value at time=0 hours.

### Results

The results are shown in Table 3, below:

The rank order of stability was therefore as follows (numbers indicate SEQ ID NOs): 16>10>12>14>1>15>11>13>5>7>8>9. Two representative HPLC traces from the stability testing are shown in FIGS. 24 and 25.

TABLE 3
Stability of dogfish glucagon and 11 analogues following incubation
with mouse plasma after 8 h. The samples were separated on
RP-HPLC and the peak height compared to the value at time = 0 h.

| | | Percentage of |
|---|---|---|
| | | degradation |
| | Peptide analogue | after 8h |
| Control | Glucagon | 29.5% |
| 5 | Dogfish glucagon (1-29) | 38.8% |
| 7 | (D-A²) dogfish glucagon | 40.1% |
| 8 | (Aib²) dogfish glucagon | 47.4% |
| 9 | (Abu²) dogfish glucagon | 58.4% |
| 10 | (D-A²) dogfish glucagon exendin end | 18.3% |
| 11 | (D-A² I⁷) dogfish glucagon | 33.0% |
| 12 | (D-A² I¹²) dogfish glucagon | 20.7% |
| 13 | (D-A² A¹³) dogfish glucagon | 33.9% |
| 14 | (D-A² D-Y¹³) dogfish glucagon | 26.5% |
| 15 | (D-A² D-D²¹) dogfish glucagon | 31.7% |
| 16 | (D-A²) dogfish glucagon-Lys³⁰-γ-glutamyl- | 11.6% |
| | PAL | |

1-22. (canceled)

23. A method of treating obesity, type-2 diabetes, or metabolic syndrome in a subject in need thereof, the method comprising: administering to a subject a therapeutically effective amount of a peptide comprising 12 to 50 amino acids, wherein the peptide includes the amino acid sequence MDNRRAK.

24. The method of claim 23, wherein the peptide comprises 20 to 45 amino acids.

25. The method of claim 24, wherein the peptide comprises 27 to 32 amino acids.

26. The method of claim 25, wherein the peptide comprises 29 amino acids.

27. The method of claim 23, wherein the peptide has the amino acid sequence of SEQ ID NO:5.

28. The method of claim 23, wherein the peptide has the amino acid sequence of SEQ ID NO:5 with one or more amino acid modifications.

29. The method of claim 28, wherein the one or more amino acid modifications comprise:
i) substitution at position 7 or 12 with isoleucine;
ii) substitution at position 13 with alanine;
iii) substitution at position 1 with tyrosine;
iv) substitution at position 2 with alanine, D-alanine, 2-aminoisobutryic acid or 2-aminobutyric acid;
v) amidation of the C-terminus;
vi) replacement of an L-form amino acid with a D-isomer equivalent; and
vii) attachment of a lipophilic moiety to a lysine residue.

30. The method of claim 29, wherein the peptide comprises a lipophilic moiety attached to a lysine residue.

31. The method of claim 30, wherein the lysine residue is the lysine residue at position 30.

32. The method of claim 29, wherein the lipophilic moiety is derived from a fatty acid.

33. The method of claim 32, wherein the fatty acid is a saturated fatty acid.

34. The method of claim 33, wherein the saturated fatty acid is palmitic acid.

35. The method of claim 29, wherein the lipophilic moiety is γ-glutamyl-palmitate.

36. The method of claim 29, wherein the peptide has a substitution at position 2 with D-alanine.

37. The method of claim 28, wherein the peptide comprises 8 or fewer amino acid modifications, wherein the modifications comprise an addition, a deletion, or a substitution, or a combination thereof.

38. The method of claim 28, wherein the peptide comprises 4 or fewer amino acid modifications, wherein the modifications comprise an addition, a deletion, a substitution, or a combination thereof.

39. The method of claim 23, wherein the peptide comprises the amino acid sequence of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18.

40. The method of claim 39, wherein the peptide comprises the amino acid sequence of SEQ ID NO:7, SEQ ID NO:16, SEQ ID NO:5, and SEQ ID NO:6.

41. The method of claim 39, wherein the peptide comprises the sequence of SEQ ID NO:7 or SEQ ID NO:16, preferably SEQ ID NO:7.

42. The method of claim 23, wherein the subject is a human subject.

43. The method of claim 23, wherein the subject is obese.

44. A pharmaceutical composition comprising a peptide comprising 12 to 50 amino acids, wherein the peptide includes the amino acid sequence MDNRRAK, and wherein the peptide does not have the amino acid sequence of SEQ ID NO:5.

45. A peptide comprising 12 to 50 amino acids, wherein the peptide includes the amino acid sequence MDNRRAK, wherein the peptide does not have the amino acid sequence of SEQ ID NO:5, and wherein the peptide stimulates insulin secretion.

46. The peptide of claim 45, wherein the peptide stimulates insulin secretion at least 30% more than does a peptide having the amino acid sequence of SEQ ID NO:5.
